(19) United States
(12) Patent Application Publication
(10) Pub. No.: US 2017/0252386 A1
(43) Pub. Date: Sep. 7, 2017

(54) METHOD FOR FEEDING POULTRY

(71) Applicants: Stanislav KHIRUG, Vantaa (FI); Sergey KHIRUG, Vantaa (FI); Alexandra VYSHTAKALIUK, Kazan (RU)

(72) Inventors: Stanislav KHIRUG, Vantaa (FI); Sergey KHIRUG, Vantaa (FI); Alexandra VYSHTAKALIUK, Kazan (RU)

(21) Appl. No.: 15/602,510

(22) Filed: May 23, 2017

Related U.S. Application Data
(63) Continuation-in-part of application No. 14/377,958, filed on Aug. 11, 2014.

(51) Int. Cl. A61K 36/21; A23K 20/174; A23K 50/75; A23K 10/30

(52) U.S. Cl. CPC A61K 36/21; A23K 10/30; A23K 20/174; A23K 50/75; A23K 10/10

(57) ABSTRACT

A method for feeding poultry is provided and includes the steps obtaining a feed supplement composition including an aqueous ethanol extract isolated from leafy parts of Amaranthus hybridus by using a mixture of water and ethanol as an extractive agent, in which mixture an amount of ethanol is within a range of 0.1-85 vol %, admixing the composition into a basal poultry feed or drinking water in a predetermined amount of at least 0.003 g/kg, and administering the resulting mixture to poultry in accordance with predetermined dosage regimen. The composition may be also utilized as a phytogenic feed supplement for poultry, depending on dosage regime, administration duration, and on age and category of poultry stock.

### FIELD OF THE INVENTION

The present invention relates generally to a method for feeding poultry by administering thereto a feed supplement composition obtained from leafy parts of Amaranthus plant, said composition being admixed into a basal poultry feed or drinking water.

### BACKGROUND

Since the complete ban on antibiotic feed additives for animal and poultry feeding in agriculture within the European Union in 2006, the interest in developing of phytogenic, i.e. of plant origin, feed additives continues to grow. It is of general knowledge that plants, especially that of medicinal or aromatic origin, contain biologically active substances, which modes of actions on the organism of living species is yet not fully studied. However, phytogenic food supplements are known to possess manifold effect on the organism, contributing to the improvement of nutritive performance of total diet and at the same time acting as bioregulators of main functional systems of the organism.

Plants of genus Amaranthus L generally known as amaranth are widely spread and well known since Aztecs Amaranth grains have been researched during last decades for particularly high squalene content in their lipid fraction. It is also known, that amaranth phytomass is rich on flavonoids, represented particularly by rutin, which is, from therapeutical point of view is powerful antioxidant and from the pharmacological point of view is important for increasing the strength of blood capillaries and regulating their permeability. Amaranthus paniculatus and A. cruentus were shown to be a particular good source for flavonoids. Studies on amaranth extracts as a diuretic agent were undertaken (Martirosyan et al, Pharmacological Properties of Amaranthus, Legacy 15:2003 (1)). Same research group had worked out the use of Amaranth in modern diet for humans and developed a technology of extracting oil from amaranth seeds by hexane.

Antimicrobial agent and composition containing extracts of plants of genus Amaranthus, namely A. caudatus, A. cruentus, for use as a food, medicine or cosmetic additive, is disclosed in JP11255612A.

U.S. Pat. No. 5,186,963 discloses a dietary composition for infants and adults containing processed seeds of plants of family Amarantaceae as a protein source, along with fats, carbohydrates and a group of vitamins. The amount of amaranth flour in said composition was 30 g per 100 ml.

Use of amaranth plant as a feed additive for farm animals is known from patent RU2374898. For the preparation of said additive amaranth oilcake has been used. The method of feeding of young chicken employing administering a vitamin additive derived from amaranth phytomass is disclosed in RU2160994; however, the method disclosed concerns merely young chickens (aged 1-40 days) of commercial layers, thereupon improved stock preservation rates and increased weight has been achieved. It was also demonstrated, that Amaranth vitamin-grass meal has stimulative effect on reproductive system development in replacement pullets and increases laying ability in laying hens in initial period of oviposition (Vyshtakalyuk et al., Agrobiology 2 (2010), 45-51).

However, there were no detailed study yet undertaken on therapeutical effects of plants of genus Amaranthus on poultry and farm livestock and no products, providing such effects are known. Regardless of that, an improvement of the efficiency and cost-effectiveness of farm animal and poultry production by means of feeding said livestock still remains to be an economic priority. It would be therefore highly advantageous to provide a product with a manifold function, which may act as a health-safe and cost-effective feed supplement, while possessing therapeutic activity, depending on the dosage regime, administration method, amount of active ingredient and the method of the production of the supplement. It would be further desirable to provide such a product, which therapeutic activity would allow using it as effective veterinary means for prevention and treatment of common diseases in poultry and farm livestock caused by increased constant stress impacts on animal organism in crowded living conditions of confinement at high stocking density. In addition, it would be desirable, that above mentioned product would be obtained from easily available raw material and by means of a process that is economical to operate.

### SUMMARY OF THE INVENTION

The present invention relates to a method for feeding poultry according to what is defined in the independent claim 1. The method comprises: (a) obtaining a feed supplement composition comprising an aqueous ethanol extract isolated from leafy parts of Amaranthus hybridus by using a mixture of water and ethanol as an extractive agent, in which mixture an amount of ethanol is within a range of 0.1-85 vol-%, (b) admixing said composition into a basal poultry feed or drinking water in an amount of at least 0.003 mg/kg, and (c) administering the resulted mixture to poultry in accordance with predetermined dosage regimen. Upon admixing the feed supplement composition obtained at step (a) into a basal poultry feed or drinking water in the mentioned amount (0.003 mg/kg of the basal poultry feed or drinking water), an effect of enhancing performance and productivity in poultry in conditions of industrial poultry farming can be stimulated, thereby the above mentioned amount constitutes a lowest effect amount for inducing desired effect(s). The definition of enhanced performance and productivity includes hereby a variety of poultry category-dependent individual effects, such as improving meat quality for meat poultry, improving egg-laying performance for egg-laying poultry, and the like.

The aforesaid feed supplement composition is referred to, in some instances, as a nutraceutical composition, or a “composition”. Professional term ‘nutraceutical’, that has been evolved recently, combines the term ‘nutrient’ and the term ‘pharmaceuticals’, and is used herein to denote utility in nutritional, pharmaceutical and veterinary fields of application. The term “nutraceutical” herein is used in order to describe the broad and manifold influence of said composition to the organism of a subject, whereupon said composition may be applied either as a nutritional supplement for subject's feed or as a substance, possessing pharmacological effects on subject's organism when administered in effective amount. The prerequisites for each particular method of application will be disclosed further. The term ‘subject’ herein, is applied in general to farm poultry. In accordance with this embodiment, the above said composition is provided in a form of a veterinary composition intended for use in prevention and/or treatment of physiological symptoms associated with at least one of an immune system disorders, digestive system disorders, nervous system dysfunctions, reproductive system disorders, erythropoiesis dysfunctions, metabolic disorders and viral infections within poultry, farm livestock, fur-bearing animals, farmed fish and household animals, fish and birds in need of such treatment, wherein the prophylactic and therapeutic effects of said nutraceutical composition on said subjects include at least one of the following: stimulation of the immune system, enhancement of food digestibility and stimulation of the digestive system, stimulation of the oogenesis, reducing cholesterol level and increasing erythrocyte level in blood and normalization of metabolic functions.

In the preferred embodiment of the invention a method for feeding poultry is provided, wherein the feed supplement composition, comprising as an active ingredient a substance, obtained from fresh, ensiled or dried phytomass of genus Amaranthus, in particular A. hybridus, by means of aqueous ethanol extraction (extraction by an aqueous solution of ethanol) is provided. In some other embodiments the composition may be obtained using other extraction methods, such as aqueous extraction or an extraction by an aqueous solution of vegetable oil. The term “aqueous extraction” herein equals to the term “hydrolysis extraction”.

The aforesaid feed supplement composition is further admixable into a basal poultry feed or drinking water, wherein the lowest effective amount thereof constitutes 0.003 promille in terms of dry feed material. The resulted mixture, referred hereby as “a phytogenic feed supplement”, can be administered to poultry in order to compensate possible deficiency of nutritional and/or vitamin components in feed of relatively healthy object. By administration of said phytogenic feed supplement to poultry at least one of the following industrial effects may be achieved: improved growth performance, improved quality of meat, improved laying ability and reproductive ability in egg-laying hens and increased livability rate. In addition, said phytogenic feed supplement contributes to the improved palatability of feed, and as a consequence—to the increased feed intake, improved feed digestibility and intensified feed conversion rate or decreased feed amount used per one unit of meat- or egg- production output.

In further embodiments of the invention a set of methods, related to the general improvement of various industrial parameters in poultry, is provided, said methods comprise administration of the above mentioned phytogenic feed supplement in accordance with an individual dosage regime, poultry breed and/or age and life cycle phase of poultry stock.

The term “extract” refers in this disclosure to a liquid product, comprising phytomass-derived complex of extractive substances dissolved in an extracting agent. The term “hydrolysate” refers in this disclosure to a suspension-like product, wherein a liquid fraction (an extract) and a residual fraction (a filtrate cake) are not separated. The term “filtrate cake”, accordingly, refers in this disclosure to a residual fraction of an extraction process. The term “regular” refers in this disclosure to the extraction products comprising little or no pectin. Pectin-containing extraction products are generally referred in this disclosure by the term “pectin-rich”. Terms “flock” and “stock” applied to poultry in this disclosure are interchangeable and both refer to experimental and/or control poultry batches. The term “dry matter” (or “dry substance”) is utilized hereby to indicate a so called air-dry (feed) material. Moisture content of said air-dry (feed) material normally constitutes about 5-8%.

Embodiments of the present invention will become apparent by consideration of the detailed description and accompanying tables.

### BRIEF DESCRIPTION OF THE DRAWINGS

FIG. 1 is a Table 1 that concerns gematological parameters for commercial layer replacement chickens, broilers and broiler breeders in absolute units (control) and in percent from the control (in batches the feed supplement composition was administered thereto).

FIG. 2 is a Table 2 concerning neutrophil activity in 46-50 days old commercial layer replacement pullets, the feed supplement composition was administered thereto starting from 4th and ending at 37th days of life, in absolute units (in the control) and in percent units (%) from the control (in batches wherein the nutraceutical composition of the invention was utilized).

FIG. 3 is a Table 3 concerning results for antibody titer to Newcastle disease study according to the La Sota method in chickens, feed supplement composition was administered thereto during the first month of raising/rearing period for no less than 2-3 weeks. Normative antibody titer parameter to Newcastle disease, characteristic of successfully formed immunodefence according to the La Sota method, in regards to which parameter the values in parenthesis were calculated, comprises 80% of vaccinated population.

FIG. 4 is a Table 4 concerning results on determination the mass of immunocompetent organs in commercial layer replacement chickens and broilers, the feed supplement composition was administered thereto.

FIG. 5 is a Table 5 concerning analysis of liver state in 42-44 days old broiler chickens, the feed supplement composition was administered thereto during raising period.

FIG. 6 is a Table 6 concerning the effect of the feed supplement composition on liver state in broilers and commercial layer replacement chickens and the ratio between healthy, conditionally healthy and cull liver in % from the overall number (control) and in % from the control (batches that have received said composition), and the effect of said composition on the mass of good liver (in absolute and relative units).

FIG. 7 is a Table 7 concerning the effect of the feed supplement composition on vitamin content in liver (by mass unit of liver in its natural state) of 40-42 days old broiler chickens.

FIG. 8 is a Table 8 concerning the effect of the feed supplement composition on biochemical blood parameters, denoted herein as a “liver functional assay”, characterized by the activity level of alanine aminotransferase (ALT) and aspartate aminotransferase (AST).

FIG. 9 is a Table 9 concerning the effect of the feed supplement composition on the development of gastrointestinal tract organs in commercial layer replacement chickens and broilers.

FIG. 10 is a Table 10 concerning the effect of the feed supplement composition on feed palatability.

FIG. 11 is a Table 11 concerning the effect of the feed supplement composition on digestibility rates of nutritional components of feed formula, wherein the experimental values are expressed in percent ratio with regard to values, corresponding to control diet.

FIG. 12 is a Table 12 concerning the effect of the feed supplement composition on a functional state of pancreas in commercial layer replacement chickens and broilers.

FIG. 13 is a Table 13 concerning the effect of the feed supplement composition on cholesterol- and triglyceride content in blood serum of commercial layer replacement chickens and broilers.

FIG. 14 is a Table 14 concerning the effect of the feed supplement composition on overall protein, albumin and urea levels in blood serum of layer replacement chickens, broilers and broiler breeders, in absolute units (control) and in % from the control level (in batches the composition was administered thereto).

FIG. 15 is a Table 15 concerning protein- and nitrogenous metabolism parameters according to the results of balance experiments in broilers that had received the feed supplement composition, in % from said parameters level in control rations without nutraceutical composition.

FIG. 16 is a Table 16 concerning the content of calcium (Ca) and phosphor (P) and the activity of alkaline phosphatase in blood serum of broilers and broiler breeders that have received the feed supplement composition in an effective amount.

FIG. 17 is a Table 17 concerning the effect of the feed supplement composition on mineral balance in broiler chickens.

FIG. 18 is a Table 18 concerning the effect of the feed supplement composition on reproductive system development in layer replacement chickens during initial egg-laying period, in % from the corresponding control batch.

FIG. 19 is a Table 19 concerning the effect of the feed supplement composition on egg-laying ability in layers during critical periods of egg-laying cycle, in percent ratio (%) from the corresponding control batch.

FIG. 20 is a Table 20 concerning parameters for reproductive performance in layer breeders and broiler breeders the feed supplement composition was administered in effective amounts thereto during egg-laying period, in % from the corresponding control batch.

FIG. 21 is a Table 21 concerning the effect of the feed supplement composition on relative heart mass, calculated in % from the normative value, comprising 0.8% of body mass.

FIG. 22 is a Table 22 concerning the effect of the feed supplement composition in the form of AH or pectin-rich AH in a dosage of 10‰ of dry matter from total mass of ration on average weight, uniformity, mortality, culls and feather pecking in layer replacement chickens. Average weight, CV %, mortality, culls and feather pecking parameters are mentioned in absolute units (control ration) and in % from the control (experimental ration).

FIG. 23 is a Table 23 concerning a comparative evaluation of anti-stress and metabolic effects caused by the feed supplement composition in broiler chickens, raised in industrial- and in vivarium conditions (both experimental), in % from the corresponding control batch.

FIG. 24 is a Table 24 concerning carcass yield in poultry, calculated as an average mass of eviscerated carcass in % from an average live weight of bird before slaughtering (control), and in % from the corresponding control batch.

FIG. 25 is a Table 25 concerning the functional condition parameters for layer breeders at the moment of slaughtering in 110 days after an establishment of the forced molt regime, wherein said molt is induced by three day starvation and darkness stress impact; and average daily preservation and productivity parameters during 72^{d}-93^{d} days of molt period, in absolute units (control ration) and in % from the control (experimental ration).

FIG. 26 is a Table 26 concerning the content of natural antioxidants in the feed supplement composition, obtained from amaranth phytomass by means of extraction (calculated by 100 g of completely dry product).

FIG. 27 is a Table 27 concerning livability and productivity parameters for the flock of broiler chickens that have received during first three days of life prophylactic preparations in the form of drinking solutions, including an antibiotic either together with the feed supplement composition in the form of aqueous extract (experimental) or without said composition (control), wherein the daily dosage was 6000 mg per kg of body mass, in % from the overall flock for preservation parameters, in grams for body mass (control 1) and in % from the control 1 (the rest of control and experimental batches). Corresponding parameters for the first control batch (control 1), that have received as prophylactic measures a standard set of three preparations such as antibiotic Colmik, vitamin complex Introvit and glucose, are considered 100%.

FIG. 28 is a Table 28 concerning livability and productivity parameters for broiler chicken flock, that have received during first three days of life the feed supplement composition in the form of aqueous extract, as preventive measures, alternative to antibiotics and vitamin complex, in % from the overall flock for preservation parameters, in grams for body mass (control batch) and in % from the control level (experimental batch).

FIG. 29 is a Table 29 concerning parameters for productivity, livability and functional state of hematopoietic system, as well as for protein, lipid and mineral metabolism, in broiler chickens, that have received the feed supplement composition in the form of an aqueous ethanol extract admixed to drinking water, during the first five days of life either in the absence of prophylactic antibiotic or jointly with it. In the control 1 batch all parameters are given in absolute values. In the rest of the batches all parameters are given in % from the corresponding values of control 1 batch. The parameters, that got worse as affected by antibiotic, are underlined.

FIG. 30 is a Table 30 concerning growth intensity and feed conversion rates parameters for broiler chickens, upon administration of the feed supplement composition as a growth promoting phytobiotic (exp. 3, 1.3 and 2.3), in % from formula parameters without antibiotics (control), or from formula parameters wherein either an in-feed antibiotic (exp. 1) or a probiotic (exp. 2) were added. All preparations were included into basic formula (BF) in effective amounts. As antibiotics (AB) Zink-bacitracin and Salinomycin were used; as a probiotic (PB)—Cellobacterin was used; as a phytobiotic (PhB)—the feed supplement composition was used in the form of an aqueous hydrolysate.

FIG. 31 is a Table 31 concerning trait parameters for various functional systems in broiler chickens, upon administration of the feed supplement composition in the form of an aqueous hydrolysate as a growth-promoting phytobiotic supplement (exp. 3 and 1.3), in % from parameters of basic formula without antibiotics (control), or from parameters of formula an in-feed antibiotic was added thereto (exp. 1). Superscripts in the “parameter” column denote that for these parameters in control batches only relative values are provided. For the rest of the parameters in control batches absolute values are provided, relatively to which parameters in experimental batches were calculated. Parameters, getting worse upon the effect of either an antibiotic or the feed supplement composition are underlined. Similarly, parameters, getting improved upon the effect of those preparations are shown in bold. In control batches also the values deviating from norm are underlined.

FIG. 32 is a Table 32 concerning the effect of the feed supplement composition on poultry livability parameters, in % from the corresponding parameters calculated for poultry on a control diet, without addition of said composition.

FIG. 33 is a Table 33 concerning a regimen for the feed supplement composition uptake into the ration of layer replacement chickens and broilers for the improvement of feed quality (palatability and assimilability).

FIG. 34 is a Table 34 concerning the effect of the feed supplement composition on growth intensity of egg-laying and meat chickens, as well as on feed conversion and slaughter meat yield, in % from the corresponding parameters calculated for poultry on a control diet, without addition of said composition.

FIG. 35 is a Table 35 concerning efficiency comparison of the feed supplement composition in combined form of aqueous ethanol extract and aqueous hydrolysate in 1:1200 ratio to separate components of said combination in corresponding amounts, in % from productivity and feed conversion parameters for poultry on a control diet, without addition of said composition.

FIG. 36 is a Table 36 concerning the effect of the feed supplement composition on live weight of broilers in different rearing periods. Live weights and flock uniformity, evaluated by coefficient of variation (CV % values in parenthesis) in control batches are given in absolute values, in experimental batches—in % from the corresponding parameters for poultry on a control diet.

FIG. 37 is a Table 37 concerning the effect of the feed supplement composition on quality of poultry meat, in % from the control ration, said composition was not administered thereto. Organoleptic properties of boiled meat and broth were evaluated in grades by five-grade scale; average value for four control and four experimental batches are outlined herein. Technological properties and chemical composition of meat are defined by the results of a single experiment.

FIG. 38 is a Table 38 concerning an administration regimen for the feed supplement composition during the experiments on the effect thereof on egg productivity in commercial layers, and breeding capacity in layer and broiler breeders.

### DETAILED DESCRIPTION OF THE INVENTION

In accordance with the preferred embodiment of the invention the method utilizes the feed supplement composition, referred to hereinbelow as a “composition”, comprising as an active ingredient a substance, obtained from phytomass of genus Amaranthus plants, is provided. For clarity purposes, further the plant of genus Amaranthus will be denoted as “amaranth”. It is preferred that leafy (green) aboveground phytomass of the amaranth are utilized (including leaves and stems, but not roots).

For the purposes of the invention it is further preferred that those amaranth plants are utilized hereby that can be cultivated as forage crop, i.e. agriculturally cultivated species of amaranth used for feeding farm animals and poultry (i.e. not wild, weed or ornamental).

Hence, the method disclosed hereby utilizes the composition comprising, as a functional ingredient, the extract obtainable only from those plant species of the genus Amaranthus that are cultivated as forage crop, but not from all plant species of said genus. This limitation is considered important, because from about 90 known species of the Amaranthus plant only 3-5 could be used as forage crops. Usability as forage crops depends on a number of factors, in particular on volume of vegetative phytomass (crop yield) obtainable from amaranth plants. Thus, the aboveground vegetative parts of the amaranth species applicable as forage crops may reach 1.5-3 m in height, whereas most weed amaranth species may reach only 10-15 cm in height.

The composition disclosed hereby has been researched within an industrial facility, such as a poultry farm; therefore, limitation of the Amaranthus species to the ones cultivated as a forage crop finds basis in applicability and usability thereof for feeding of poultry/farm livestock in conditions of industrial farming.

At present it has been shown that the most of amaranth species cultivated as forage crops are subspecies of A. hybridus, therefore they have a common origin. For example, according to the Agricultural Research Service available at the webpages of United States Department of Agriculture (ARS), subspecies of Amaranthus hybridus L. include: Amaranthus hybridus L. subsp. cruentus (L.) Thell. (referred as a synonym to Amaranthus cruentus L.); and Amaranthus hybridus L. var. hypochondriacus (L.) B. L. Rob. (referred to as a synonym to Amaranthus hypochondriacus L.).

Hence, chemical composition of mentioned amaranths would be similar. From the other hand, it is known that vegetative parts (phytomass) of the Amaranthus plants contain a variety of biologically active substances of largely undefined nature. Respectively, functional effects thereof have been studied only superficially. Hence, the present invention application aims at solving this problem and provides the amaranth phytomass based product having both nutritional and therapeutic value and demonstrating a variety of simultaneously occurring functional effects on different groups of poultry and/or other farm animals.

In preferred embodiment Amaranthus hybridus plants are utilized.

Said biologically active substance (or substances) comprises biologically active compounds of amaranth and is obtained from amaranth phytomass by means of extraction, preferably, by means of an aqueous ethanol extraction. Fresh, dried or ensiled amaranth phytomass may be utilized as a crude material for the extraction process. The following extracting agents may be utilized: water, whey, aqueous solutions of acids or alkali, aqueous solutions of cellulase enzymes, oil-water emulsion and ethanol-water solutions. Both conventional and modern techniques may be utilized to implement an extraction process, such as rotary-pulsation-, pressurized-liquid extraction, sub- and supercritical-fluid extraction, microwave-assisted-, ultrasonic- extraction techniques and the like.

The feed supplement composition thus comprises the aqueous ethanol extract isolated from leafy parts of Amaranthus hybridus ssp. by using a mixture of water and ethanol as an extractive agent, in which mixture an amount of ethanol is within a range of 0.5-70 vol-%. Said extract may be provided as a liquid fraction selected from the group consisting of:
an entire fraction isolated by using the mixture of water and ethanol as an extractive agent, in which an amount of ethanol is about 0.1 vol-%, said fraction optionally containing pectin compounds;
a sediment-free supernatant fraction isolated by using the mixture of water and ethanol as an extractive agent, in which an amount of ethanol is about 0.1 vol-%, said fraction optionally containing pectin compounds;
a sediment-free supernatant fraction isolated by using the mixture of water and ethanol as an extractive agent, in which an amount of ethanol is about 70 vol-%, wherein upon isolating the pectin-containing fractions aqueous solutions of whey, acids and/or cellulase enzymes are utilized as additional extractive agents; and wherein upon isolating the essentially pectin-less fractions, water and aqueous solutions of alkali are utilized as additional extractive agents.

A feed supplement composition (nutraceutical composition) disclosed hereby may comprise an active substance, obtained from amaranth phytomass either separately, as a supernatant fraction of the extraction process, herein, an extract, or together with the residual fraction of the extraction process, herein, a hydrolysate. Said nutraceutical composition may thus be utilized in liquid-, substantially semi-solid, e.g. as a suspension, and in substantially solid, e.g. dried, forms. The amount of dry substance in the extract is about 30% from that in the hydrolysate.

The hydrolysate fraction, obtained by an aqueous extraction process, may comprise predominantly proteins, pectins or saccharides, or the complex of these products in various combinations.

The feed supplement composition disclosed hereby may be provided in the following formulation, which may be classified as disclosed below.
1. The feed supplement composition comprising regular aqueous extraction products, obtained as a result of an extraction by water or aqueous solutions of alkali in the form of:
   aqueous hydrolysate (AH);
   aqueous extract (AE);
2. The feed supplement composition comprising pectin-rich aqueous extraction products obtained as a result of an extraction by whey and/or aqueous solutions of acids and alkali, and by whey and/or aqueous solutions of acids, alkali and cellulase enzymes in the form of:
   pectin-rich aqueous hydrolysate (pectin-rich AH);
   pectin-rich aqueous extract (pectin-rich AE).
3. The feed supplement composition comprising aqueous oil extraction products obtained as a result of an extraction by aqueous solutions of vegetable oil in the form of:
   aqueous oil hydrolysate (AOH);
   aqueous oil extract (AOE).
4. The feed supplement composition obtained as a result of an extraction by aqueous solutions of ethanol in the form of aqueous ethanol extract (AEE).

A method for feeding poultry is provided hereby comprising: (a) obtaining a feed supplement composition comprising an aqueous ethanol extract isolated from leafy parts of Amaranthus hybridus by using a mixture of water and ethanol as an extractive agent, in which mixture an amount of ethanol is within a range of 0.1-85 vol-%; (b) admixing said composition into a basal poultry feed or drinking water in an amount of at least 0.003 mg/kg in terms of the basal poultry feed or drinking water; and (c) administering the resulted mixture to poultry in accordance with predetermined dosage regimen.

In some preferred embodiment the feed supplement composition obtained at step (a) is provided in the form of a liquid preparation and comprises the extract isolated by using a mixture of water and ethanol, in which mixture amount of ethanol is about 0.5 vol-%.

In another preferred embodiment the feed supplement composition obtained at step (a) is provided in the form of a dry preparation provided as a powder or a particulate, said composition comprises the extract isolated by using a mixture of water and ethanol, in which mixture amount of ethanol is about 70 vol-%.

In still another embodiment the dry feed supplement composition obtained at step (a) is further admixed to a suitable liquid carrier prior to performing the step (b). Suitable liquid carrier may include water, any suitable solvent, suspension, emulsion, and the like.

In some embodiment, the dosage regimen (step c) includes administering the feed supplement composition, obtained at step (a) as a dry preparation and further admixed to the suitable liquid carrier, to poultry via drinking water in daily amount of 0.2-6 g per kg of live weight.

In some embodiment, the dosage regimen (step c) includes administering the feed supplement composition, obtained at step (a) as a liquid preparation and comprising the extract isolated by using a mixture of water and ethanol, in which mixture amount of ethanol is about 0.5 vol-%, to poultry via drinking water in daily amount of 2.7-6 g per kg of live weight.

In some embodiment, the dosage regiment (step c) includes administering the feed supplement composition, obtained at step (a) as a dry preparation and comprising the extract isolated by using a mixture of water and ethanol, in which mixture amount of ethanol is about 70 vol-%, to poultry via the basal feed formula in daily amount of 0.3-60 mg, preferably 0.3-5.4 mg, per kg of live weight.

In some embodiment, the method comprising administering the feed supplement composition to poultry selected from the group consisting of: broilers, breeders, commercial layers at different age stage, and replacement chickens of breeder- and commercial layer categories.

In some embodiment the dosage regimen (step c) includes administering the feed supplement composition, obtained at step (a) as a dry preparation and comprising the extract isolated by using a mixture of water and ethanol, in which mixture amount of ethanol is about 70 vol-%, to broiler chickens continuously during individual rearing periods, in particular, from the first day of life until slaughtering via the basal feed formula in daily amount of 0.3-60 mg, preferably 0.3-5.4 mg, per kg of live weight, as a substitution for in-feed antibiotics and coccidiostats. Upon observing the above indicated dosage regimen the following effect are obtainable: improved growth performance, increased live-weight gain attainable by an enhanced feed palatability and an increased feed intake, improved feed conversion efficiency attainable by increased digestibility rates of feed nutrients, and enhanced eviscerated carcass yield and improved meat quality, attainable by reduced stress impact.

In some embodiment the dosage regimen (step c) includes administering the feed supplement composition, obtained at step (a) as a dry preparation and further admixed to the suitable liquid carrier, to broiler chickens during the first 3-5 days of life via drinking water in daily amount of 0.2-6 g per kg of live weight, such as to improve growth performance, and increase a live-weight gain, attainable by increased digestibility rates of feed nutrients.

In some embodiment the dosage regimen (step c) includes administering the feed supplement composition, obtained at step (a) as a liquid preparation and comprising the extract isolated by using a mixture of water and ethanol, in which mixture amount of ethanol is about 0.5 vol-%, to replacement chickens and commercial layers at different age stages continuously during individual rearing periods or in short-term periods of 10-20 days via the basal feed formula in daily amount of 9-360 mg, preferably 9-108 mg, per kg of live weight, such as to improve a laying performance and to enhance feed conversion efficiency, attainable by increased digestibility rates of feed nutrients.

In some embodiment, the dosage regimen (step c) includes administering the feed supplement composition, obtained at step (a) as a liquid preparation and comprising the extract isolated by using a mixture of water and ethanol, in which mixture amount of ethanol is about 0.5 vol-%, to layer breeders and broiler breeders at different age stages and to the corresponding replacement chickens continuously during individual rearing periods or in short-term periods of 10-20 days via the basal feed formula in daily amount of 9-360 mg, preferably 36-60 mg, per kg of live weight, such as to improve a settable egg quality, to enhance feed conversion efficiency and to prevent disease outbreaks, feather pecking and cannibalism in poultry in conditions of stress impact during catching, moving and vaccination, and to improve reproductive performance after feed withdrawal induced molting.

The classification above is utilized for the disclosure of the particular formulations, generalized by the term “feed supplement composition” (“nutraceutical composition” or “composition”), in further examples.

For those skilled in the art it is clear that a composition, comprising a substance, obtained from natural, i.e. plant source and containing biologically active compounds may not be strictly considered as a drug. However, it is also clear, that said biologically active compounds may naturally have a regulatory effect on the functional systems of an organism, thus possessing possible curative and protective properties.

The composition obtained from Amaranthus phytomass, as disclosed hereby, may thus be utilized as a curative and protective food supplement, which purpose is to supply nutritional components otherwise lacking from the feed ration, but it may be also utilized as means for the prevention and treatment of certain physiological conditions caused by dysfunctions of the organism. Dual nature of said composition is thus expressed by the term “nutraceutical”. The term “veterinary preparation” will be applied when referring to use of the composition for prevention and treatment further disclosed.

The use of the composition predominantly either as a phytogenic feed supplement or as a veterinary preparation is predefined by the extraction method and thus the content of the composition, the dosage regime and the duration of administration. The term “phytogenic feed supplement” thus refers to a feed supplement of a plant origin, obtained from amaranth in this disclosure.

The composition was studied in farm poultry, wherein term “farm poultry” denotes at least one of the following strains of chickens: commercial layers of White Leghorn P-46; layer breeders White Leghorn P-4 and P-6; replacement chickens comprising commercial layer replacement pullets and cockerels of White Leghorn P-46, layer breeder replacement pullets and cockerels of White Leghorn P-6 and P-4, broiler breeder replacement chickens of Hubbard Flex; broiler breeders of Hubbard Flex; broiler chickens of Hubbard Flex, Hubbard F15, Hubbard JV. Above mentioned farm poultry had been kept in cages. The composition, however, is suitable for administration to any other category of farm poultry, and, in addition, also to farm livestock, such as cattle, swine, sheep and/or goat, to fur-bearing animals, farmed fish and to household animals, fish and birds.

For the purposes of the invention, the composition is admixed to basal feed formula (infeed) of farm poultry and the other above mentioned subjects in the following way. The composition in the form of regular aqueous or pectin-rich aqueous extraction products (AH, pectin-rich AH, AE, pectin-rich AE) extracts may be introduced to feed formula in daily doses equal to 7.5-360 mg per kg of live weight. The composition in the form of AE or pectin-rich AE may be admixed to drinking water (in-water) and administered as a drinking solution in an amount equivalent to that, introduced to feed formula. Alternatively the composition may be administered first in short-term (3-5 days) in daily doses 514-4000 mg per kg of live weight, followed by in-feed introduction in daily doses 7.5-360 mg/kg. In an initial growth period and for the infectious diseases preventive care, the intensive dosage regime may be implemented, wherein the composition in the form of AE may be administered in daily doses 2700-16000 mg/kg for 3-5 days. The composition in the form of AOH may be introduced to feed formula in a daily dose of about 54 mg/kg. The composition in the form of AEE pre-diluted in water may be introduced to feed formula in daily doses 0.05-60 mg of dry matter per kg of live weight. AEE may be admixed to drinking water and administered in-water in an amount equivalent to that, introduced to feed formula, or, alternatively, administered during an initial growth period for 3-5 days in daily doses 0.7-25 mg/kg, followed by introducing AEE to feed formula in daily doses 0.05-5.4 mg/kg. In the initial growth period and for infectious diseases preventive care, AEE as a drinking solution may be administered for 3-5 days in daily doses 200-7000 mg/kg. The composition, comprising a mixture of AEE and AH in 1:400-1:1200 ratio by dry weight, may be introduced to feed formula in daily doses 0.1:36-0.3:36 mg/kg.

The approaches undertaken to study preventive and therapeutic effects of the composition on various functional systems of the organism of farm poultry are disclosed below. All experiments concerning farm poultry were conducted in the conditions of industrial facilities, wherein poultry birds experience constant stress, related to the intensive bird-keeping technology. Keeping conditions for control and experimental poultry batches differed only by presence or absence of the nutraceutical composition in the ration (diet). The product efficiency was evaluated on the background of real functional condition of poultry, which more or less differed from the physiological standards. Whether the condition parameters of some functional systems of the poultry organism were in physiological standards limits, the optimized performance of a functional system was considered as a measure for prophylactic action of the nutraceutical composition. Whether same system parameters were beyond the physiological standards limits, the normalizing action of the composition may be considered as therapeutic.

For the evaluation of the effects of nutraceutical composition on various systems of poultry organism functioning, certain biochemical, immunological and hematological blood parameters were investigated, as well as internal organs condition by means of visual evaluation, weighting and histological study thereof. Histological study comprised cutting paraffin sections 7-10 μm thick, hematoxylin-eosin dyeing thereof and microscopic studies (Carl Zeiss, zoom 150, 300 and 600). The blood for analysis was withdrawn either during lifetime, from brachial wing vein, or during slaughtering by exsanguination. Blood analysis was performed for no less than 5 birds from each batch. After slaughtering, the condition of internal organs was investigated for the same birds.

In accordance with some embodiments, an administration of the nutraceutical composition in order to cause positive effects on the functional systems of poultry organism may be complied in accordance with the following formulations.

For the correction of hematopoiesis disorders and anemia prevention pectin-rich aqueous extraction products or AEE are preferably utilized.

For the correction of immunodeficiency conditions related to cell-mediated immunity disorders pectin-rich aqueous extraction products are preferably utilized.

For the strengthening of humoral immunity, for immunity formation during vaccination, for the improvement of functional condition of spleen and for the inhibition of thymus and bursa shrinking the nutraceutical composition in the form of any of the above mentioned extraction products may be utilized.

For the prevention and treatment of reproductive system disorders, nutraceutical composition, comprising extractive substances obtained from amaranth phytomass by aqueous or aqueous ethanol extraction, are utilized.

For the prevention and treatment of liver disorders, such as hepatitis, colibacillosis, liver avitaminosis and increased in size liver, a nutraceutical composition in the form of AH, AE and AEE are utilized.

For the prevention and treatment of diseases and dysfunctions of the pancreatic gland, such as increased content of blood amylase and pancreatic gland hyperplasia, a nutraceutical composition in the form of AEE or in the form of pectin-rich AH and pectin-rich AE are utilized.

For the prevention and treatment of weakened function of the digestive system, herein depressed feed digestibility and decreased appetite, the nutraceutical composition in the form of any of the above mentioned extraction products may be utilized.

For the stimulation of the gastrointestinal tract development, the nutraceutical composition in the form of pectin-rich AH is preferably utilized.

For the prevention and treatment of lipid metabolism malfunctions and for the reduction of cholesterol level, for the prevention and treatment of protein and nitrogen metabolism disorders, herein reducing urea level in blood and increasing the level of blood albumin, and mineral metabolism disorders, accompanied by calcium, phosphorus and other minerals assimilation malfunctions and bone tissue mineralization abnormalities, the nutraceutical compositions comprising any aqueous extraction products, AEE and a combination of AEE and AH in 1:400-1200 ratio by dry weight may be utilized.

For the prevention and treatment of mostly widespread infectious diseases for farm animals and poultry during their initial rearing period, the nutraceutical composition in the form of any of the above mentioned extraction products, may be utilized. During individual rearing periods the nutraceutical composition may be applied as a growth-promoting phytobiotic substitute to in-feed antibiotics.

Experiments that confirm functionality and action of the nutraceutical composition, were conducted mostly on farm poultry. Poultry breeds and the administration- and dosage regimes are disclosed below.

The terms “broiler” and “broiler breeder” in any combination refers in this disclosure to meat poultry breeds. The terms “layer” and “layer breeder” refer in this disclosure to egg-laying poultry breeds. The term “replacement chickens” is utilized hereby to indicate both pullets and cockerels more than 40 days old, but has not yet entered maturity age.

For the broiler chickens, in order to improve feed conversion and stock uniformity rate thereof, as well as to increase mass gain and to reduce mortality rates and negative stress-induced effects, the nutraceutical composition is administered in accordance with the following regimen:
either during the whole rearing period, starting from the 1^{st} day of life till slaughtering (39-45 days), or during certain rearing stages at least one of the following formulations is introduced into the feed formula (in-feed): AE or pectin-rich AE—in daily doses 0.075-0.72‰ (7.5-72 mg/kg); AH or pectin-rich AH—in daily doses 0.3-16‰ (9-480 mg/kg), and AOH—in daily doses 1.8‰ (54 mg/kg). Instead of in-feed administration extracts may be admixed to drinking water in equivalent to that of in-feed amounts;
in a time frame from the 1^{st} to 3-9^{th} days of life either AE is introduced to drinking water (in-water) in daily doses 2700-16000 MΓ/KΓ, or AEE—in daily doses 200-7000 mg/kg;
in a time frame from the 1⁴ to 3-7^{th} days of life AE is introduced to drinking water in daily doses 514-4000 mg/kg; followed by, starting from 8-14^{th} days of life to 28-45^{th} days of life, in-feed daily introduction of AH in an amount 0.3-12‰,l AE in an amount of 0.09-0.15‰, AOH in an amount of 1.8‰ or a combination of AEE and AH in 1:400-1:1200 ratio by dry weight in an amount of 0.001:1.2‰. Average daily dose of in-feed introduced dry extractive compounds comprises 9-360 mg/kg;
in a time frame from the 1⁴ to 3^{d} days of life AEE is introduced in-water in doses 0.7-25 mg/kg, followed by, starting from 14^{th} day of life till slaughtering, in-feed AEE introduction in doses 0.0005-0.054‰ (0.05-5.4 mg/kg);
in a time frame from the 21^{st} to 45^{th} days of life AEE is introduced daily into feed formula in an amount of 0.6‰ (60 mg/kg).

For the replacement chickens, including commercial layer pullets and breeder layer replacement pullets and cockerels during the time frame from the 1^{st} to 120^{th} days of life, in order to stimulate a maturation of reproductive system, to improve stock uniformity, to increase feed conversion rates, to reduce negative stress-induced effects, to improve livability, to reduce mortality rates and incidences of cannibalism, the nutraceutical composition is administered in accordance with the following regimen:
either during the whole rearing period or during certain rearing stages at least one of the following formulations is daily introduced into the feed formula: AE or pectin-rich AE in an amount of 0.09-2‰ (9-200 mg/kg), or AH or pectin-rich AH in an amount of 1-12‰ (30-360 mg/kg);
in a time frame from the 1^{st} to 3-9^{th} days of life either AE or pectin-rich AE is in-water introduced daily in dose 6000 mg/kg;
in a time frame from the 70^{th} to 110^{th} days of life during 7-14 days at least one of the following formulations is daily introduced into the feed formula: AH or pectin-rich AH in an amount of 5-10‰ (150-300 mg/kg), AE or pectin-rich AE in an amount of 1.5-3‰ (150-300 mg/kg), or AEE in an amount of 0.4-2.2‰ (40-220 mg/kg).

For the commercial layers, in order to stimulate a laying ability, to reduce negative stress-induced effects, to reduce incidences of pecking and cannibalism, to improve stock uniformity, to increase feed conversion rates, to improve livability and to reduce mortality rates, the composition is administered in accordance with the following regimen:
either before and during the whole laying period or during certain stages thereof at least one of the following formulations is introduced in-feed: AH or pectin-rich AH in an amounts of 1.2-3.0‰, AE or pectin-rich AE in an amounts of 0.36-0.6‰ (36-60 mg/kg).

For the layer breeders and broiler breeders, in order to improve a breeding efficiency, to enhance rates of fertilization and hatchability of settable eggs, to increase laying ability and settable eggs laying period, as well as to reduce negative stress-induced effects, to reduce incidences of pecking and cannibalism, to improve livability and to reduce mortality rates, including that after force molting, the nutraceutical composition is administered in accordance with the following regimen:

either before and during the whole laying period or during certain stages thereof at least one of the following formulations is introduced in-feed: AH or pectin-rich AH in an amounts of 1.2-2.0‰, AE or pectin-rich AE in an amounts of 0.36-0.6‰ (36-60 mg/kg).

The versatility of the invention, disclosed herein, and its manifold effects to distinct functional systems of poultry organism makes it hardly possible to be described in general. For this reason the invention will be further described by reference to the following detailed examples, which are exemplary and are intended to teach those skilled in art to utilize the invention in a most accomplished way within the scope defined by the claims thereof.

For the clarity purposes, the content of said nutraceutical composition in the feed formula is expressed in terms of promille (‰), or one part per thousand, calculated per amount of dry matter contained in hydrolysate or extract; and the daily dose of said composition, expressed in milligrams per kilogram of body mass, refers to dry content of extractive substances in extracts and hydrolysates, either upon administering as a drinking solution or upon feeding. Because nutraceutical composition in the form of hydrolysates comprises about 30% of extractive substances and the rest of it is non-filtered phytomass, the equal dosage of the extractive substances for hydrolysates and extracts is ensured by three-fold excess of hydrolysate in poultry ration in comparison to extract.

The data tables in all accompanying figures contain asterisks (from 1 to 3), that denote three different significance levels of probability (p) value for statistical hypothesis testing: 0.05, 0.01 and 0.001, correspondingly. In all data tables, unless otherwise stated, corresponding parameters for the control batch are taken as 100%; those parameters that differed from norm are underlined, and parameters, getting improved upon the effect of the nutraceutical composition are shown in bold.

### Example 1

### Correction of Hematopoietic Disorders and Prevention of the Development of Anemia with Various Etiologies

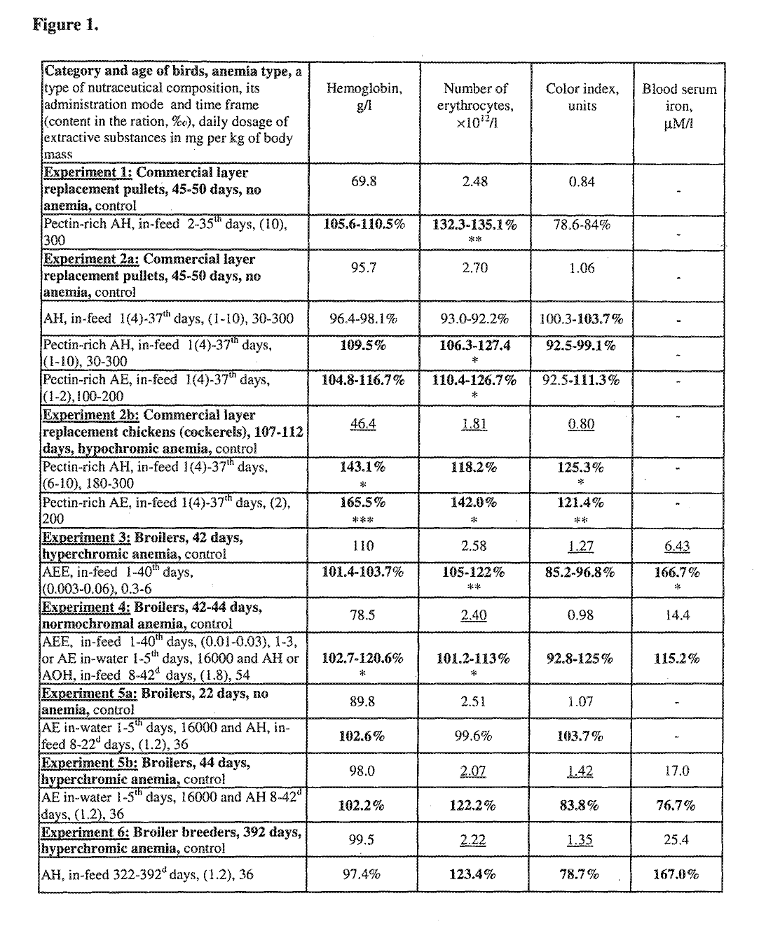

The effect of the feed supplement composition (nutraceutical composition) on the hematopoiesis system was estimated by hemoglobin content in blood, which content was determined by hemoglobin-cyanide method, by the number of erythrocytes, counted in Bürker chamber, by calculated content of hemoglobin in a single erythrocyte and by an iron content in blood serum measured on the biochemical analyzer Daytona Randox using a reagent set from Randox. The results for hematological parameters study in gallinaceous poultry birds are shown in Table 1 of FIG. 1.

In accordance with Table 1 of FIG. 1, upon administration of the nutraceutical composition to gallinaceous poultry birds, all hematological parameters, otherwise impaired in the conditions of intensive bird keeping, are improved.

At early age, when hematological parameters in chickens are in physiological standards limits, an administration of the nutraceutical composition either promotes an increase in hemoglobin concentration and in erythrocyte number, thus lowering the color index (exp. 1, table 1, FIG. 1), or does not have any significant effect on hematological parameters (exp. 2a and 5a, table 1, FIG. 1). However, a hematopoietic process in gallinaceous poultry birds of egg-laying and meat crosses gets impaired with aging. In replacement chickens of egg-laying crosses (cockerels 107-112 days old) a hypochromic anemia is developed (exp. 2b, table 1, FIG. 1), and in broilers a hyperchromic anemia is developed by a 44-days age (exp. 5b, table 1, FIG. 1). The administration of the nutraceutical composition eliminates the signs of hematopoietic dysfunction and improves hematological parameters by reducing thereof (exp. 2b, table 1, FIG. 1), as well as normalizes an increased color index by bringing the number of erythrocytes up to standard level (exp. 5b, table 1. FIG. 1). Although in broilers this effect is developed during a continuous use of said nutraceutical composition, in layer breeder replacement chickens it has a nature of an aftereffect, developing in 70 days after the end of administration.

In broilers with normochromic anemia, accompanied with the decrease in erythrocyte number to less than 2.5×10¹²/l (exp. 4, table 1, FIG. 1), as well as in layer breeder replacement chickens with hypochromic anemia, proceeding with the reduction of all hematological parameters, the nutraceutical composition causes full restoration of hematopoietic process to its normative parameters. Upon hyperchromic anemia (exp. 3 and 6, table 1, FIG. 1), occurring most often in birds with intensified plastic metabolism, such as broilers and breeder hens, and accompanied by an increase in color index up to 1.15 and over, as well as reduced erythrocyte number, the nutraceutical composition promotes an increase in erythrocyte number and normalization (lowering) of color index. Normalization of iron content in blood serum, as provided by metabolic requirements for birds, occurs upon administration of said nutraceutical composition. Whether iron content in blood serum of broilers was low (exp. 3, table 1, FIG. 1), or remained within the low limit of physiological standard (exp. 4, table 1, FIG. 1), its rise had occurred. Whether iron content in blood serum was high (exp. 5b, table 1, FIG. 1), its reduction in the limits of physiological standard had occurred, accompanied by a proportional rise in erythrocyte number (to 22%), thus reaching the norm. That implies that under the effect of said nutraceutical composition a significant intensification of hematopoiesis process occurs, eliminating hyperchromic anemia by means of more intensive consumption of iron stocks in the organism.

In those breeder hens, whose iron concentration in blood serum was determined at the end of the productive period, when the intensity of metabolic processes, as well as iron consumption for body weight maintenance and egg-laying process become less intensive, a hyperchromic anemia was developed while iron level in the blood serum remained normal (exp. 6, table 1, FIG. 1). Upon administration of the nutraceutical composition, iron content in blood serum increased to 66.7% in these hens that led not only to the replenishment of iron stocks in the body, but to the intensification of hematopoietic process and elimination of anemia.

### Example 2

### Correction of Immunodeficiency States Related to Cellular and Humoral Immunity Disorders and Immunocompetent Organs Shrinking

An effect of the feed supplement composition (nutraceutical composition) on the immune system was evaluated according to the following parameters:
1. Functional state of neutrophils, determined by spontaneous and induced neutrophil hemiluminescence, stimulated by zymosan, their ratio and response time.
2. Determination of antibody titer to Newcastle disease after chicken vaccination.
3. Weight determination of immunocompetent organs, such as thymus, bursa and spleen, investigation of histological structure of the spleen.

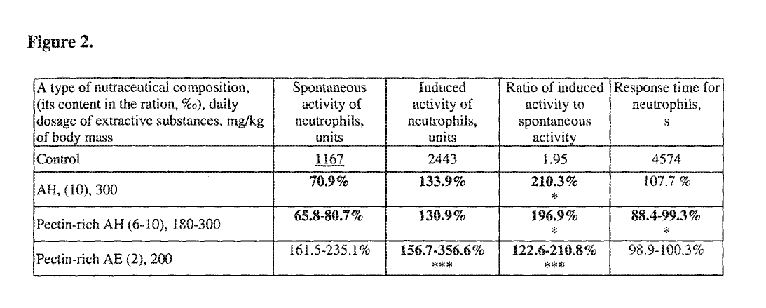

The results for cellular and humoral immunity parameters for chickens are shown in tables 2-4 of FIGS. 2-4, respectively.

As shown in Table 2 of FIG. 2, spontaneous hemiluminescent activity of neutrophils in the control batch of birds was sufficiently high, that may be due to the vaccination, conducted two weeks before the investigation. In all batches, that have received the nutraceutical composition in the form of hydrolysates, a reduction in spontaneous neutrophil activity was recorded, that may be indicative of the completion of immune response formation to antigens, that have been introduced upon vaccination, and of the increase in efficiency of the antigen inactivation process.

Induced activity of neutrophils, characteristic of the ability of cells to response to stimulants (foreign substances), and the ratio between induced and spontaneous activities of neutrophils, specifying the functional state of immunocompetent cells, i.e. their ability to response to foreign substances, increases upon the administration of said nutraceutical composition (table 2, FIG. 2).

Neutrophil response time to zymosan stimulation in chickens, that received nutraceutical composition in the form of pectin-rich AH, had decreased, that is also indicative of the functional state improvement of immunocompetent cells that become more sensitive to antigens.

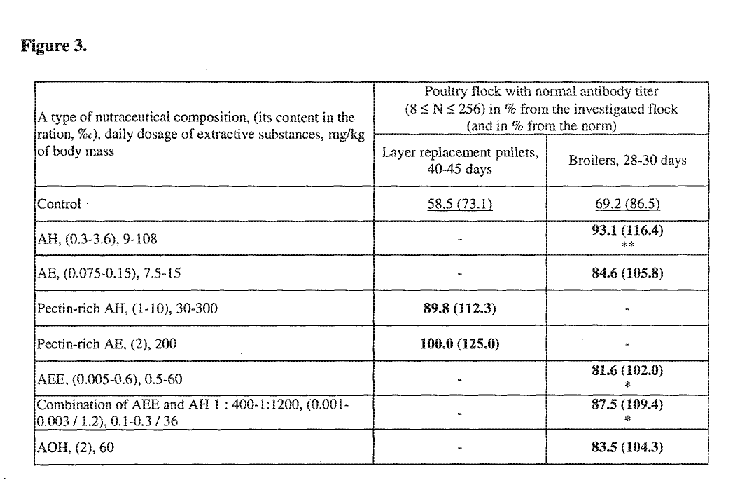

The results for the immune state study in chickens according to the La Sota method are shown in Table 3 of FIG. 3. In accordance with Table 3, in the control batches only 58.5-69.2% of chickens had antibody titer level complied with the norm, that is significantly lower comparative to normative values characteristic of the effective vaccination (80%). In all experimental batches, wherein chickens have received the nutraceutical composition, the amount of chickens with normal antibody titer reached the normative values (80%), and in one of the batches this value was even 100%. The data thus obtained indicate that said nutraceutical composition promotes more effective formation of humoral immunodefence to Newcastle disease on 14^{th} day after vaccination.

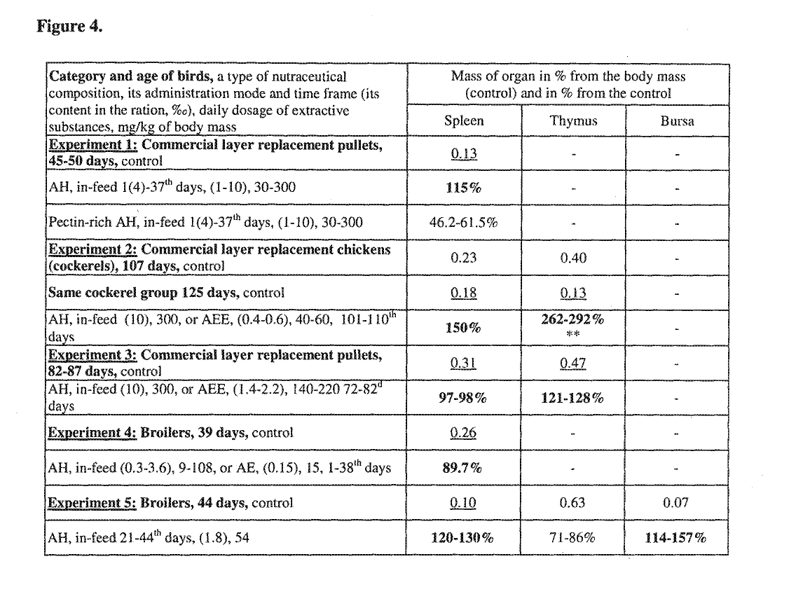

Table 4 of FIG. 4 shows the results on mass determination for the immunocompetent organs, such as spleen, thymus and bursa, in chickens. According to Table 4, an improvement of morphofunctional state of immunocompetent organs, as well as acceleration of said organ's development, and retardation in reduction and involution processes in immunocompetent lymphoid tissues were observed in chickens upon administration of the nutraceutical composition thereto.

In the control batch of 45-50 days old pullets (exp. 1, table 4, FIG. 4) and 44 days old broilers (exp. 5, table 4, FIG. 4) a decrease in relative spleen mass below norm was observed. In the control batch of cockerels of 125 days old (exp.2, table 4, FIG. 4) a decrease in relative spleen mass by 21.7% in comparison to chickens of 107 days old was observed, that may be indicative of the partial convolution of organ's lymphoid tissue within the period between two measurements. As affected by administered nutraceutical composition, the spleen mass increased in experimental batches of chickens, that may depend on the acceleration of development, or retardation in involution of immunocompetent lymphoid tissue of said organ. The histological sections of spleen in experimental batches of chickens reveal more pronounced structural pattern and moderate expansion of lymphoid tissue hyperplasia in comparison to the control batch.

The relative spleen mass in the control batches of 82 days old pullets (exp. 3, table 4, FIG. 4) and of 38 days old broilers (exp. 4, table 4, FIG. 4) reaches the upper limit of norm. During histological study of spleen of these chickens the signs of mucoid swelling in the walls of central artery and of moderate edema in perivascular connective tissue were observed. The nutraceutical composition thus promotes a decrease in spleen mass and causes the pathological changes, otherwise observed in control batches, to disappear.

The thymus mass in the control batch of cockerels of cross P-46 (exp. 2, table 4, FIG. 4) within the period from 107 to 125 days of life have decreased 67.5% that may depend on thymus lymphoid tissue involution. In those cockerels that have received the nutraceutical composition, the shrinking of thymus slowed down and comprised only 5-15% within same period (exp. 2, table 4, FIG. 4). The shrinking of thymus in 82 days old pullets effected by said nutraceutical composition had also slowed down (exp. 3, table 4, FIG. 4).

The bursa mass in 44 days old broiler chickens (exp. 5, table 4, FIG. 4), that have received the nutraceutical composition, appears to be higher than in control, that is indicative of the shrinking process of B-lymphocyte producing immunocompetent tissues slowing down.

### Example 3

### Prevention and Treatment of Liver Disorders, Such as Hepatitis, Colibacillosis, Avitaminosis and Hypertrophy of Liver

The experiments disclosed in this example include the following methods:
1) weighting the liver and gallbladder;
2) analyzing the biochemical blood parameters, such as alanine aminotransferase (ALT), aspartate aminotransferase (AST);
3) visual evaluation and sorting of the liver, obtained during industrial slaughtering of the large livestock of poultry birds (up to 3000 head in a batch).

The effect of the feed supplement composition (nutraceutical composition) on liver state is illustrated by tables 5-8. Herein, by the term ‘conditionally healthy liver’ a liver with insignificant pathological changes, such as avitaminosis, primary stages of hepatitis and liver hypertrophy, is denoted. By the term of ‘rejected liver’ a liver with pronounced pathological changes, such as hepatitis, colibacillosis, hypertrophy, is denoted. By the term of ‘good liver’ healthy and conditionally healthy liver is denoted, that was not rejected on the conveyor.

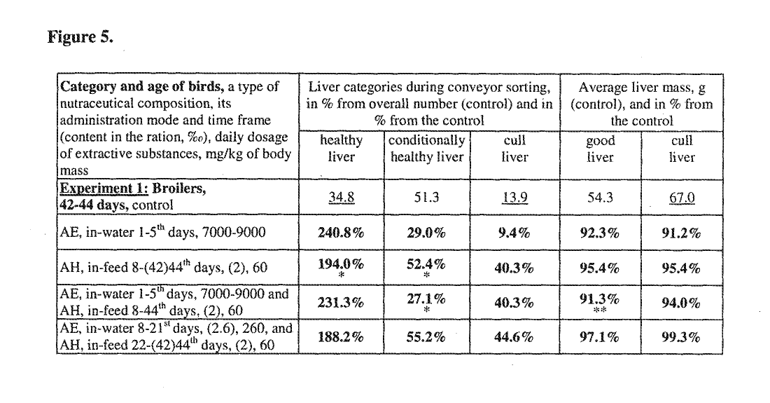

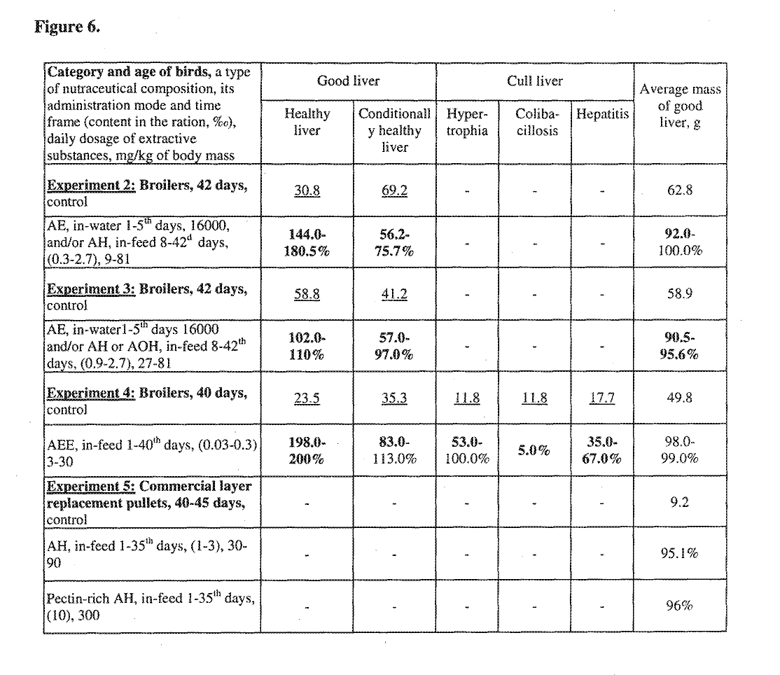

According to tables 5 and 6 of FIGS. 5 and 6, respectively, study of liver state in 40-44 days old broiler chickens indicates, that by the moment of slaughtering only 23.5-59% of chickens had clinically healthy liver (exp. 1, table 5, FIG. 5 and exp. 2, 3 and 4, table 6, FIG. 6). The number of chickens in poultry stock with clinically healthy by the moment of slaughtering liver increases 1.6-2.4 times upon administration of the nutraceutical composition. At the same time, no tendency to age-specific impairment of liver state was found at the end of the broiler rearing period (within 40-44 days).

The nutraceutical composition promotes the decrease in liver average mass (exp.1, table 5, FIG. 5). In the experimental batches an average mass of good liver statistically reliably decreased by 3-9%. By ‘good’ liver both healthy and fairly healthy liver are denoted herein. The mass of rejected liver thus decreased 0.7-8.8%. In the experiments 2, 3, 4 and 5 an average liver mass decreased by 1-6% (table 6, FIG. 6). According to the decrease in average liver mass promoted by the nutraceutical composition, the effect of said composition may be estimated as positive, directed to the normalization of liver function, improvement of poultry functional condition and increasing its biological potential.

The frequency of liver hypertrophia cases in gallinaceous poultry birds the nutraceutical composition was administered thereto was reduced almost twice; the frequency of hepatitis cases—1.5-3.fold, and of colibacillosis cases—20-fold (table 6, FIG. 6).

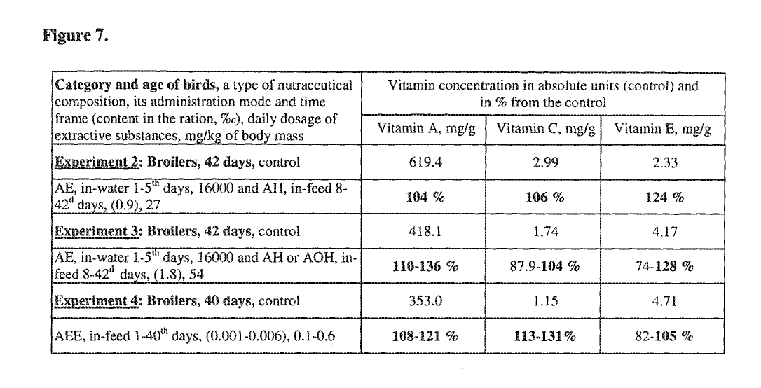

Table 7 of FIG. 7 shows the results on vitamin amount studies in poultry liver upon administration of the nutraceutical composition. According to table 7, the administration of said composition promotes an increase of vitamin A content in liver by 4-36%, an increase of vitamin E—by 5-28%, and an increase of vitamin C—by 4-31%.

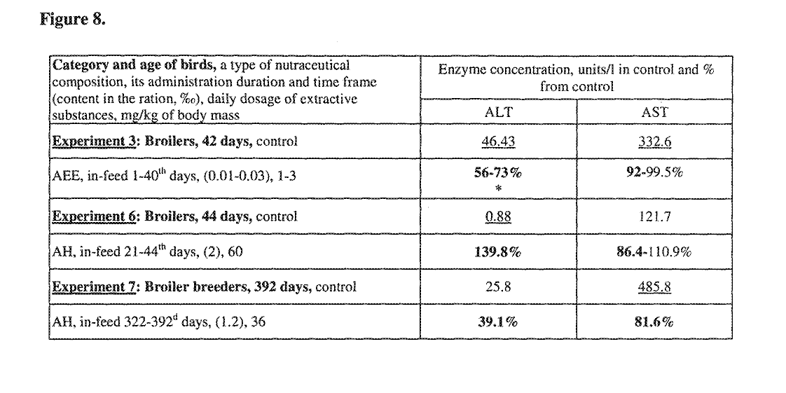

Table 8 of FIG. 8 shows that the nutraceutical composition promotes the reduction of ALT and AST enzyme levels in blood, otherwise elevated in conditions when liver experiences functional stress (exp. 3 and 7, table 8, FIG. 8), thus causing an improvement in liver functional state and lowering the intensity of pathological degenerative processes in liver tissue, which processes otherwise cause the release of said enzymes in blood. On the contrary, in conditions of decreased liver function when ALT level in control is significantly below the norm (exp. 6, table 8, FIG. 8), the administration of the nutraceutical composition promotes an intensification in said enzyme's activity.

### Example 4

### Prevention and Treatment of the Digestive System Disorders, Accompanied by the Impairment of the Digestive Function, Expressed in Reduced Feed Assimilability and Low Appetite

The experiments disclosed in this example include the following methods:
1) weighting the organs of the digestive system, such as proventriculus, gizzard, intestine and measuring the length of the intestine;
2) determination of feed consumption by head per day;
3) determination of digestibility, or assimilability, of nutritional components of the ration (dry matter, organic matter, cellulose, protein, lipids, nitrogen-free extractive compounds), by keeping an account during the balance experiment on an amount of compounds consumed with feed and excreted with excrements, and making calculations according to formula 1. The nitrogen content in bird's excrements was determined by separation the nitrogen contained in urine and excrements by means of boiling 1 g of weighted excrements in 40% of alkali (NaOH or KOH), filtration and subsequent determination of nitrogen in the residue.

$$The digestibility ( assimilability ) coeffiecient of the nutritional component ( DC ) \\ DC ( \% ) = \frac{( A - B ) \times 100}{A} . \qquad Formula 1$$

Wherein: A—is an amount of the matter, consumed with the feed,
B—is an amount of the matter, excreted with excrements,
C—is an amount of the matter, excreted with urine.

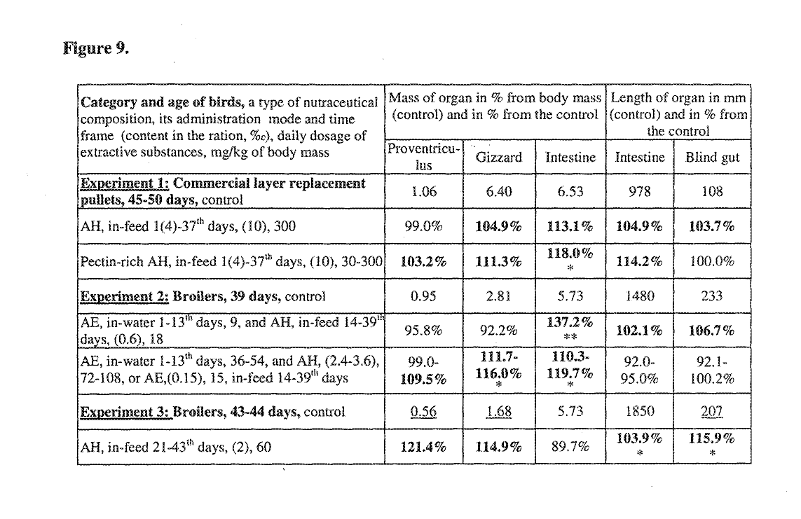

The results are shown in tables 9-11 of FIGS. 9-11, respectively. Table 9 of FIG. 9 shows, that the feed supplement composition (nutraceutical composition) stimulates development of the digestive organs, promoting an increase in proventriculus and gizzard mass, as well as of an intestine (absolute mass and with respect to body mass); and promoting the growth of intestine and blind gut in length.

The development of the digestive tract organs leads to an intestinal tract suction surface expansion, and consequently to an increase in its suction capacity. Intensified development of the digestive tract smooth muscles (gizzard muscles, for example) leads to intensified digestion process and improved motor-evacuation function. During histological investigation of intestinal tract organs the signs of growing secretory activity of digestive glands were detected in experimental batches.

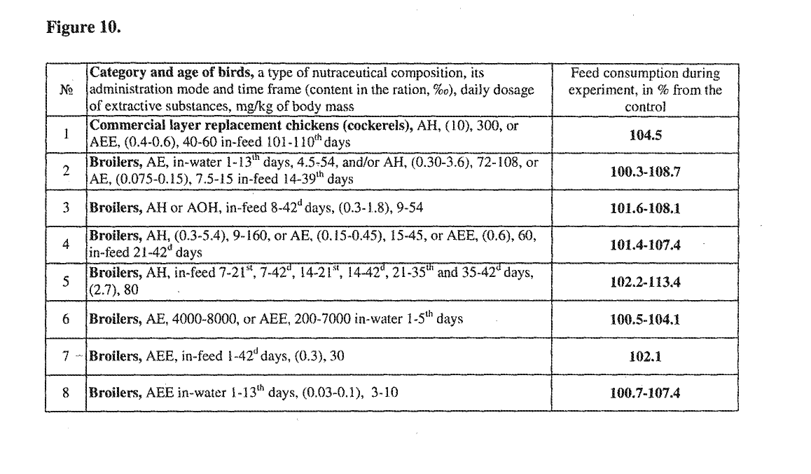

Upon the feed consumption analysis an increase in feed intake was observed in batches the nutraceutical composition was administered thereto (table 10, FIG. 10). These data indicate, that said composition improves feed palatability and promotes an increase of appetite in poultry birds.

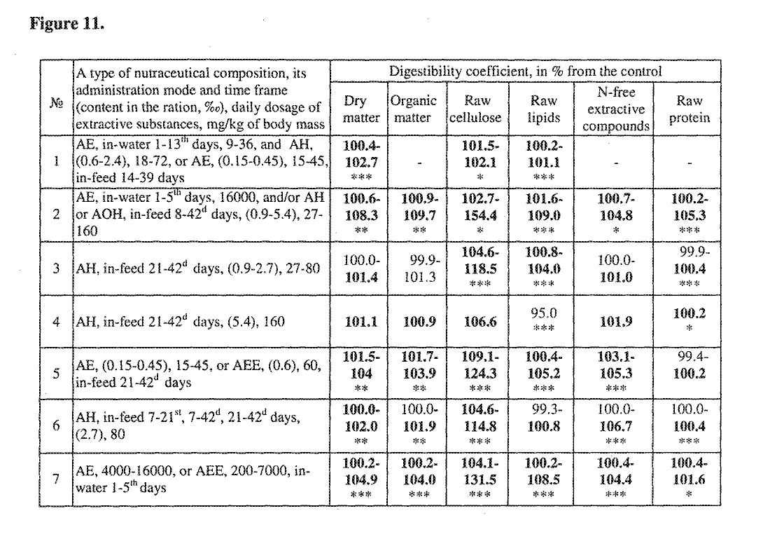

Upon analysis of the results of balance experiments, elevated digestibility rates for almost all studied nutritional components, as well as a significant increase in assimilability of basic feed under the effect of said nutritional composition were observed (table 11, FIG. 11).

The results obtained are indicative of the pronounced stimulating effect of the nutraceutical composition on the development and functional state of the digestive system.

### Example 5

### Prevention and Treatment of Pancreatic Gland Disorders

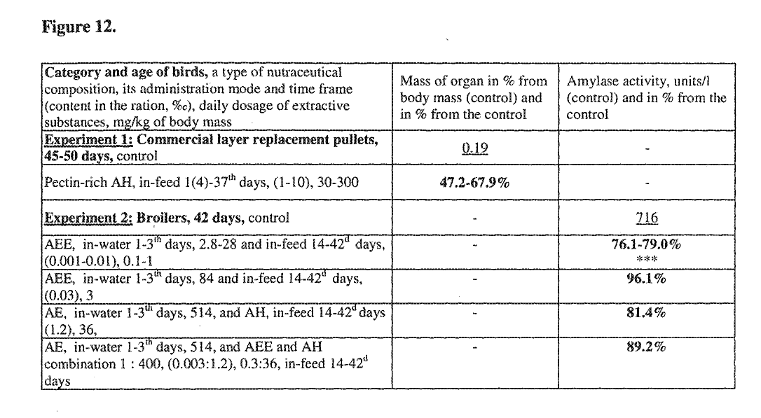

For the evaluation of the effects the feed supplement composition (nutraceutical composition) causes in pancreatic gland said composition was applied in effective amounts in accordance to that disclosed previously in this document. The condition of the pancreas was evaluated by means of weight determination and estimation the activity levels of blood serum amylase. The results are shown in Table 12 of FIG. 12.

The pancreas mass in control pullets of egg-laying crosses was sufficiently above normal (0.19% of body mass instead of 0.08-0.12%), but upon administration of the nutraceutical composition pancreas mass decreased to norm (exp. 1, table 12, FIG. 12). Control broiler chickens, in accordance with amylase activity levels in their blood serum, had pancreas in significantly worse functional condition, in comparison to that in replacement chickens of egg-laying crosses, since nutraceutical composition promoted only partial normalization of amylase activity level (exp. 2, table 12, FIG. 12), which remained considerably higher than norm (28-100 units/1). According to the results above, the nutraceutical composition may be effectively used for prevention and treatment of pancreatic gland dysfunctions.

### Example 6

### Prevention and Treatment Of Lipid (Cholesterol) Metabolic Disorders that are Accompanied by Elevated Levels of Cholesterol and Triglycerides in Blood

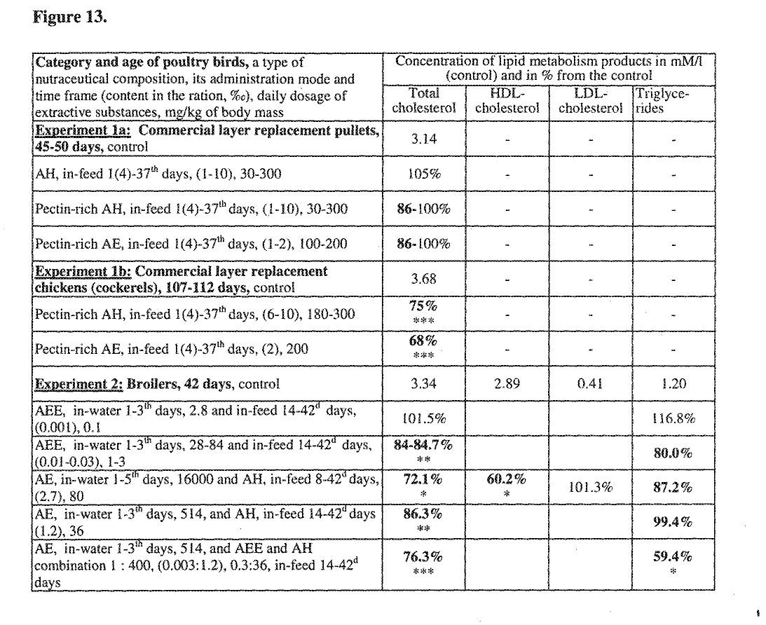

For the evaluation of the effects the feed supplement composition (nutraceutical composition) has on metabolism, in particular, on cholesterol levels in blood, said composition was applied in effective amounts in accordance to that disclosed previously in this document. On the completion of experiments blood for analysis was taken during slaughtering by means of exsanguination method. Triglyceride-, total cholesterol- and high-density lipoprotein (HDL) content was determined in serum samples. Low-density lipoprotein (LDL) content was determined by calculation methods. The results are shown in Table 13 of FIG. 13.

The effect of the nutraceutical composition on the cholesterol content in blood in commercial layer replacement chickens, which chickens neither possess fast increase in body mass nor associated metabolism dysfunctions, is pronounced very weakly during the first rearing period (till 45 days). And, the aqueous hydrolysate did not have any effect at all on chickens of said age group, although pectin-containing hydrolysates or extracts lowered cholesterol level only by 14% (exp. 1a, table 13, FIG. 13).

However, a prolonged anticholesteremic, i.e. promoting a reduction of cholesterol levels in blood, effect was observed in the same chicken batches in 62 days after the completion of administration of pectin-containing compositions (exp. 1b, table 13, FIG. 13), said effect opposing the age-related increase of cholesterol level in blood. In the experiments 1a and 1b between control batches the cholesterol content had risen by 21.7% within same time period.

In broilers, which are generally characterized by an intensive increase in body mass in short terms, metabolism dysfunctions of various kind may occur, in particular, dysfunctions in lipid metabolism. Cholesterol level in blood of control batch broilers was higher in comparison to that of replacement chickens of egg-laying crosses of similar age (45-50 days), although did not overcome the limits of physiological standard. The content of triglycerides and high- and low density lipoprotein-cholesterol was also in physiological standard limits (exp. 2, table 13, FIG. 13). Upon administration of the nutraceutical composition, also in the form of aqueous, not enriched with pectin, hydrolysate, a significant reduction in cholesterol-, triglyceride- and high density lipoprotein-cholesterol contents was observed.

Results thus obtained are indicative of that the nutraceutical composition provides a prolonged preventive action on the cholesterol and lipoprotein levels in blood and prevents the development of age-related impairments of lipid metabolism in chickens of egg-laying and meat crosses.

### Example 7

### Prevention and Treatment of Protein and Nitrogenous Metabolic Disorders that are Accompanied by Protein Level Reduction and Increased Levels of Urea in Blood Serum

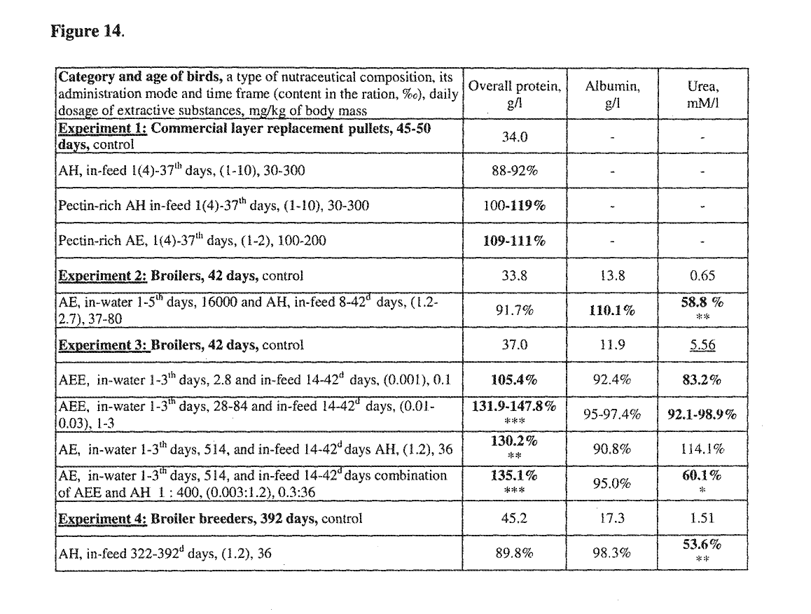

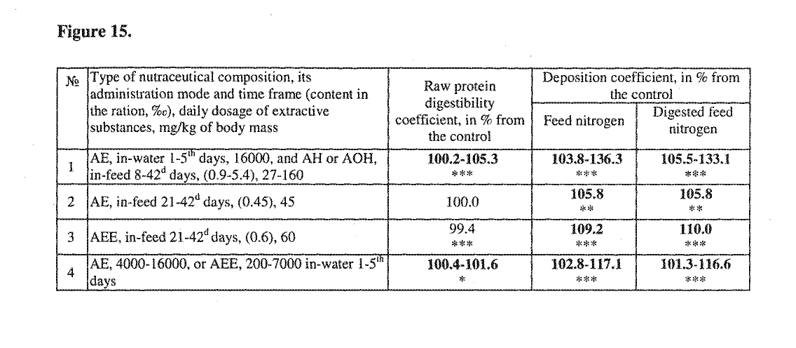

For the evaluation of the effects the feed supplement composition (nutraceutical composition) has on metabolism, in particular, on protein and nitrogenous metabolism, said composition was applied in effective amounts in accordance to that disclosed previously in this document. The experiments were conducted on layer replacement chickens, broilers and broiler breeders. At the completion of feeding period, the balance experiments were conducted on broiler chickens to study nitrogenous and protein metabolism, such as determination of raw protein digestibility, as well as fodder nitrogen and digested nitrogen utilization (deposition) coefficients (formulae 2-4). On the completion of these experiments the blood was collected for analysis by means of exsanguination method during slaughtering. The levels of overall protein, albumin and urea were determined. In addition, the protein content in pectoral and femoral muscles of broilers and in albumens of settable eggs was determined. The results are shown in tables 14 and 15 of FIGS. 14 and 15, respectively.

$$\begin{matrix}The digestibility ( assimilability ) coeffiecient of raw protein ( DC ) \\ DC ( \% ) = \frac{( A - B ) \times 100}{A} . & Formula 2 \\ The coefficient of the utilization ( deposition ) of fodder nitrogen ( CC ) \\ CC ( \% ) = \frac{( A - B - C ) \times 100}{A} . & Formula 3 \\ The coefficient of the utilization ( deposition ) of digested nitrogen ( UC ) \\ UC ( \% ) = \frac{( A - B - C ) \times 100}{( A - B )} . & Formula 4\end{matrix}$$

Wherein: A—is an amount of the nitrogen, consumed with the feed,
B—is an amount of the nitrogen, excreted with excrements,
C—is an amount of the nitrogen, excreted with urine.

In replacement chickens, such as commercial replacement chickens and broilers, the overall protein and albumin fraction contents in blood serum remain in the limits of the physiological standard (exp. 1-3, table 14, FIG. 14). Affected by the nutraceutical composition these parameters may rise significantly, although the composition in the form of aqueous hydrolysate does not contain enough pectin and is, usually, less effective (exp. 1 and 2, table 14, FIG. 14).

The overall protein- and albumin fraction contents in blood serum of breeders were reduced upon the effect of the aqueous hydrolysate (exp. 4, table 14, FIG. 14). However, meanwhile in the experimental batch protein takeaway with eggs was increased by 16.4%, and the effectiveness of fodder protein utilization for the industrial egg-production was increased by 15.8%. This indicates that the nutraceutical composition promotes more effective utilization of protein stocks of the organism, providing an increase in farm poultry productivity.

The significant reduction in urea levels in blood serum, observed in all experiments of table 14 (FIG. 14), is indicative of that the nutraceutical composition causes the slow-down in protein catabolism and intensification of anabolic processes of protein synthesis during building up the muscle bulk in broiler chickens and during egg formation in layers.

In balance experiments on broiler chickens, that have received the nutraceutical composition starting from the first day of life, a significant rise in digestibility rates of feed formula proteins, as well as an increase in feed formula nitrogen utilization (deposition) and digested nitrogen coefficients was observed (table 15, FIG. 15). If said nutraceutical composition was administered starting from the age of three weeks, the feed formula nitrogen consumption and digested nitrogen coefficients increased in less extent, and the feed digestibility rate either did not change or even slightly decreased (table 15, FIG. 15). These results are confirmed by the direct measurements of protein content in the products, obtained upon the administration of the nutraceutical composition. Thus, the protein content in broiler red meat increased by 2.1% and in albumens of settable eggs—by 2.4% (p<0.01).

Results obtained are indicative of that the nutraceutical composition accelerates the digestibility of fodder raw protein, promotes the normalization of protein- and nitrogenous metabolism, stimulates anabolic processes of protein synthesis and provides more effective utilization of proteins during industrial production of poultry meat and eggs.

### Example 8

### Prevention and Treatment of Mineral Metabolic Disorders Accompanied by Impairments in Calcium and Other Minerals Assimilation and in Bone Tissue Mineralization

For the evaluation of the effects the feed supplement composition (nutraceutical composition) has on metabolism, in particular, on mineral metabolism, said composition was applied in effective amounts in accordance to that disclosed previously in this document. The evaluation of mineral metabolism was conducted on the basis of balance experiments and blood parameters. In balance experiments the utilization (deposition) coefficients of ash, calcium (Ca) and phosphorus (P), were determined (formula 5).

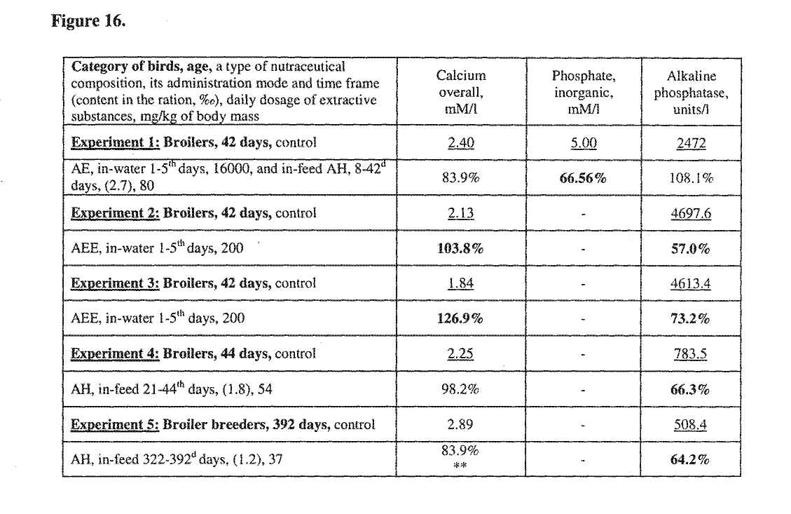

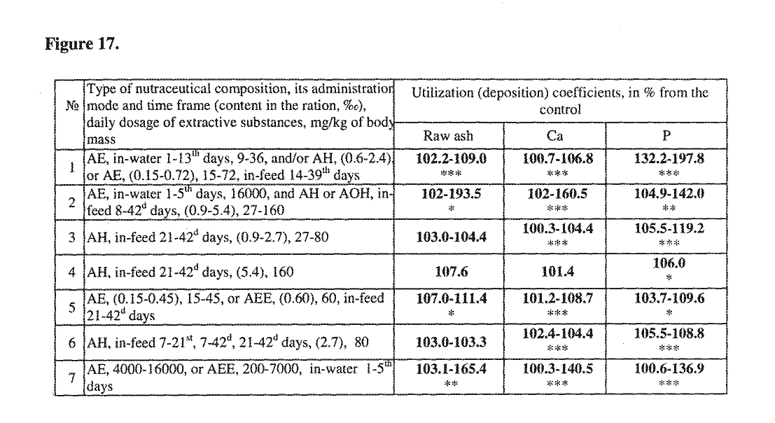

After experiments were complete the blood for analyses was collected by means of exsanguination method, at a time of slaughtering. The amount of calcium and phosphorus, as well as the activity of alkaline phosphatase in blood serum was determined. The amount of Ca, P and ash was also determined in pectoral and femoral muscles of broilers. The results are shown in tables 16 and 17 of FIGS. 16 and 17, respectively.

$$The utilization ( deposition ) coefficient of ash , calcium ( Ca ) or phosphorus ( P ) ( CC ) \\ CC ( \% ) = \frac{( A - B - C ) \times 100}{A} . \qquad Formula 5$$

Wherein: A—is an amount of the ash, calcium (Ca) or phosphorus (P), consumed with the feed,
B—is an amount of the ash, calcium (Ca) or phosphorus (P), excreted with excrements,
C—is an amount of the ash, calcium (Ca) or phosphorus (P), excreted with urine.

The lack of Ca in tissues and organs may occur even in presence of normal amount of this element in blood, in case the balance between Ca and inorganic phosphate, which content in poultry blood serum normally is 2.0 and 1.76 mM/l, is disturbed. High level of inorganic phosphate in blood (hyperphosphatemia) leads to chronic lack of Ca in the organism and is accompanied by the significant rise of alkaline phosphatase levels, that normally comprise 500 units/1 (exp. 1, table 16, FIG. 16). Administration of said nutraceutical composition in the course of the experiment caused normalization of mineral metabolism, as provided by the significant reduction of inorganic phosphate in blood serum. Moderately reduced in the limits of physiological standards calcium level in blood serum and an increased activity of alkaline phosphatase (exp. 1, table 16, FIG. 16) are due to intensified consumption of Ca by tissues and organs, lacking this element.

Significant increase in alkaline phosphatase on the background of normal Ca in blood serum (exp. 2, table 16, FIG. 16) is also indicative of a mineral metabolic disorder, related to the lack of Ca. Upon administration of the nutraceutical composition, a normalization of alkaline phosphatase levels, as well as slight increase in Ca content in blood serum had occurred, that is indicative of the reduced Ca deficiency in broiler chickens. In case of more profound lack of Ca in the organism, when its contents in blood are reduced below the limits of physiological standard, and alkaline phosphatase levels exceed the norm manifold (exp. 3, table 16, FIG. 16), said nutraceutical composition provides an essential increase of Ca levels in blood serum.

In those cases, when Ca content in broilers and breeders was in the limits of physiological standards, and the alkaline phosphatase activity exceeded the norm limits only insignificantly (exp. 4 and 5, table 16, FIG. 16), upon administration of said nutraceutical composition calcium concentration decreased and the alkaline phosphatase activity was normalized. Thus, in conditions of balanced mineral nutrition the nutraceutical composition may provide more intensive consumption of Ca stocks in order to maintain necessary levels of bone mineralization in broilers, and to support egg formation in breeder hens. This fact is confirmed in broilers, which have received the nutraceutical composition, by the increased ash content in white meat by 8.2%, by the increased amount of calcium in red meat by 50%, phosphorus in white meat—by 1%, and by the increased bone mass relative to body Mass—by 9%.

The analysis of balance experiment's results indicates that those broiler chickens that have received the nutraceutical composition demonstrate an improved mineral balance, in particular, increased utilization (deposition) coefficients for raw ash, Ca and P (table 17, FIG. 17).

The data obtained are indicative of the role said nutraceutical composition plays in normalization of mineral metabolic disorders with various degrees of profoundness, in the improvement of mineral nutrition and in more effective utilization of mineral compounds, in particular Ca and P, for the formation of organs and tissues of growing broiler chickens. Thus, said composition may be used for the prevention and treatment of mineral metabolic disorders and corresponding diseases in farm poultry in the conditions of the industrial farming.

### Example 9

### Prevention and Treatment of Reproductive System Functional Disorders, Associated with Age-Related Changes and Intensive Bird-Keeping Technology

An effect of the feed supplement composition (nutraceutical composition) on functional disorders of the reproductive system in poultry is best to study during critical periods of egg-laying cycle, upon formation of reproductive organs or upon age-related changes in reproductive system function, such as ovulation, fertilization, egg formation and embryo development. One of such periods, related to poultry keeping technology, is a force molt, during which process a rearrangement of the reproductive system occurs, thus providing a transition to a new egg-laying cycle.

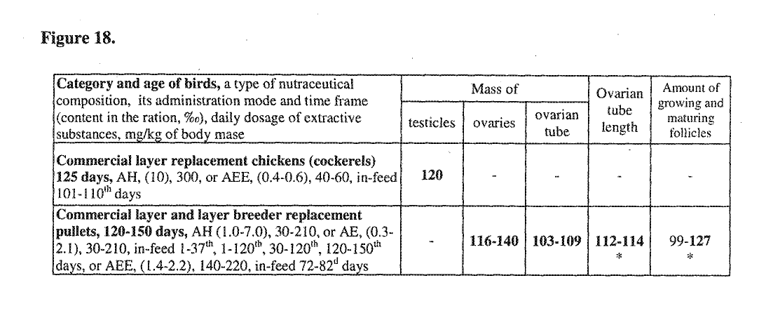

For the evaluation of the effects the nutraceutical composition has on reproductive system, said composition was applied in effective amounts in accordance to that disclosed previously in this document. The effect of said composition on the functional condition of reproductive system in layers was evaluated by the development of reproductive organs (mass of ovary and ovarian tube, length of ovarian tube), histological composition of ovary, amount of laid eggs, by parameters of embryonic development, condition and viability of one day old chicks. The egg-laying process intensity defines a functional condition of ovary and an efficiency of hormonal regulation that ensures ovulation intensity. Embryonic and post-embryonic development of new hatched chicks, dependent on the quality of settable eggs, defines the functional condition of reproductive system organs, in which egg formation takes place. The effect of said composition on rooster's reproductive system functional condition was evaluated by the development of testicles (mass of said organ with respect to body mass) and by breeding efficiency (amount of unfertilized eggs with respect to the overall number of settable eggs). In order to investigate the condition of reproductive organs after the completion of the experiments, 5-8 birds from each batch were slaughtered by means of exsanguination method. Experimental results, as well as product application sheets are shown in tables 18-20 of FIGS. 18-20, respectively.

Studies were conducted during the following critical periods of egg-laying cycle: 1) beginning of the egg-laying period in young pullets, when organs of the reproductive system are being formed; 2) in hens, at the end of a force molt period, when the reproductive system function is being restored; 3) in grown-up hens, in the completion phase of egg-laying period, when age-related dysfunction of the reproductive system occurs, causing the decrease in egg-laying intensity, fertility rates and hatchability.

As a result of the conducted study it was shown, that the nutraceutical composition causes an increase in mass of the reproductive system organs both in pullets and in cockerels, which is indicative of the stimulating effect of said composition on the development of their reproductive system (table 18, FIG. 18). During histological study of the ovary in those batches of young egg-laying pullets, that had received said nutraceutical composition, an increased number of maturing follicles and more intensive growth of interstitial tissue was recorded compared to that in control, which is indicative of the enhancement of ovary function under the effect of said composition.

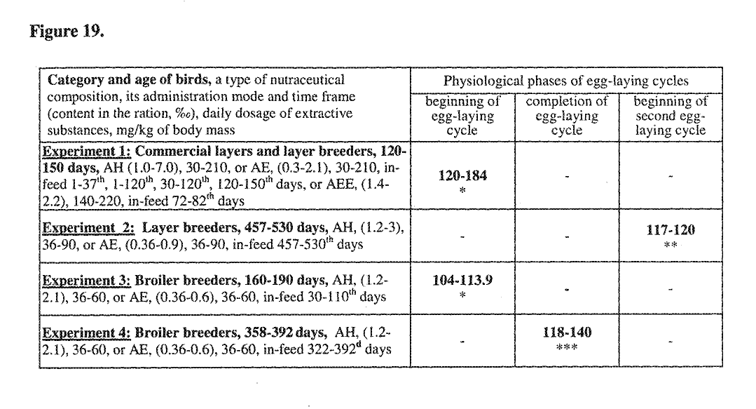

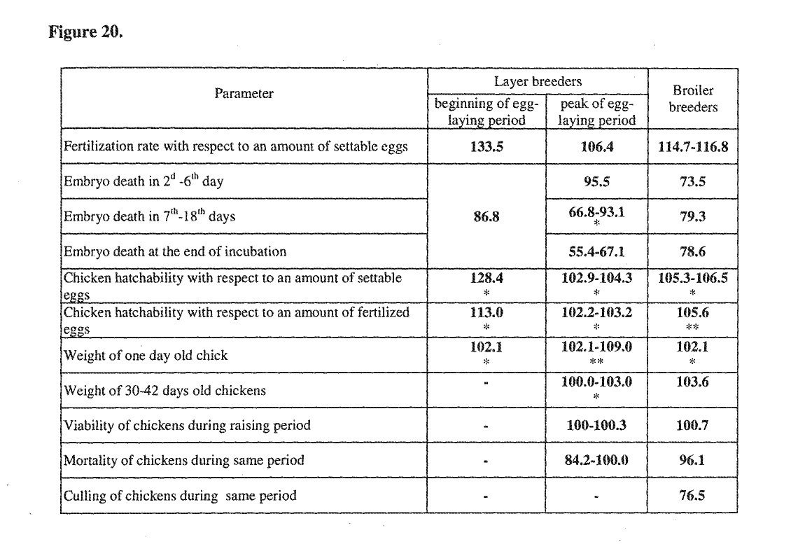

During the critical periods of egg-laying cycle a substantial increase in an amount of laid eggs occurs upon administration of the nutraceutical composition that is indicative of the stimulating effect said composition has on reproductive system development. It is as well indicative of a possibility of restoration of disturbed egg-laying cycle by utilizing said composition (table 19, FIG. 19). Also, a decreased amount of unfertilized eggs indicates sperm quality enhancement in roosters. In addition, embryonic mortality on different development stages is reduced, chicken hatchability rate is increased, and the weight, viability and growth intensity of newly hatched chickens are improved (table 20, FIG. 20).

Results thus obtained indicate that, an administration of the nutraceutical composition causes a significant improvement in functional condition of the reproductive system in breeders, settable egg quality enhancement and an increased viability of next generation in embryonic and post-embryonic development stages. These processes facilitate an elimination of functional disorders of the reproductive system that occur during critical periods of egg-laying cycle and are associated to age-related changes in birds and intensive technology of industrial bird keeping.

### Example 10

### Correction of cardiovascular system related disorders.

In conditions of industrial farm animal- and poultry keeping, wherein a maximum productivity is aimed for, conditions for the intensive gain of body mass are created, thus accompanied by stress and metabolic dysfunctions that result in turn in cardiovascular system disorders. One of the indicators for such disorders is a heart mass deviation from the norm, which may be caused by heart dysfunction. In chickens the heart mass normally comprises 0.8% from the body mass.

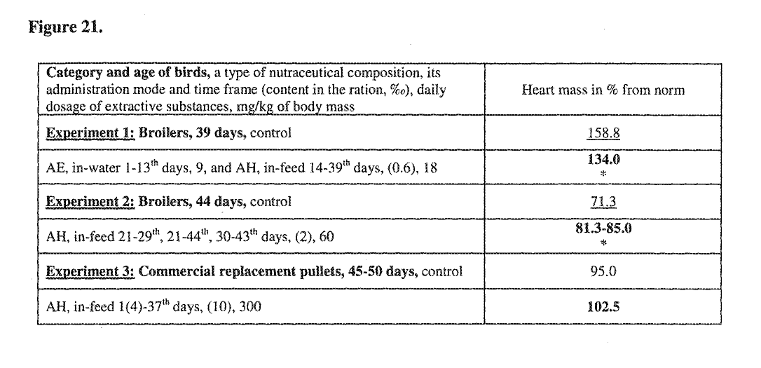

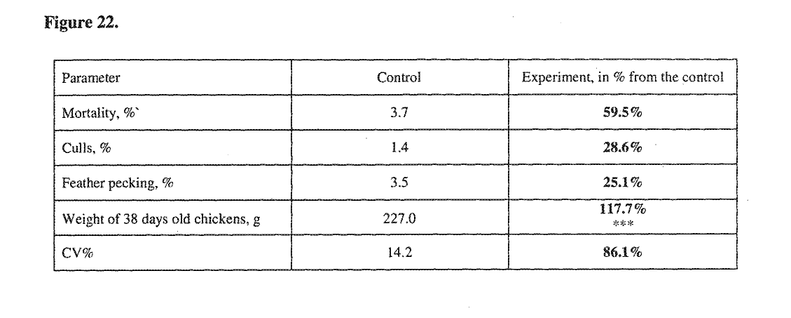

For the evaluation of the effects the feed supplement composition (nutraceutical composition) has on the heart condition, said composition was applied in effective amounts in accordance to that disclosed previously in this document. In order to evaluate the heart condition after the completion of the experiments 5-8 birds from each batch were slaughtered by means of exsanguination method, the absolute mass of heart was determined by weighting and heart mass with respect to body mass was calculated. Experimental results, as well as product application sheets are shown in Table 21 of FIG. 21.

The most pronounced heart mass deviations from norm were identified in broilers, which may have either hypertrophy or atrophy of a cardiac muscle (table 21, FIG. 21). Said poultry group is characterized by the most intensive body weight gain, thereupon having a high risk to develop disturbances in a cardiovascular system performance

Cardiac muscle hypertrophy may be caused by increased functional stress, leading in broilers to compensatory disturbances in a cardiovascular system performance Upon the administration of the nutraceutical composition such disturbances occur less often, that is expressed in substantially reduced hypertrophy of a cardiac muscle (exp. 1, table 21, FIG. 21). During the late rearing periods in broilers, when the compensatory capacity of the bird's organism is already pushed to the limit, weakening of a cardiac muscle may lead to its atrophy (exp.2, table 21, FIG. 21). In these conditions the positive effect of said nutraceutical composition is expressed in partial restoration of cardiac muscle mass. Thus, in both cases under the effect of said nutraceutical composition heart mass in broilers approaches the norm that is indicative of functional stress reduction, otherwise leading to cardiovascular system dysfunctions.

Heart mass deviation from the norm in replacement chickens of egg-laying lines was substantially lower than in broilers, and that was the reason for almost complete restoration of cardiac muscle mass upon administration of said nutraceutical composition thereto (exp. 3, table 21, FIG. 21).

Data thus obtained confirm that the nutraceutical composition may be effectively utilized for the prevention and treatment of cardiovascular system related disorders in farm poultry.

### Example 11

### Preventive Maintenance of Nervous- and Endocrine System Related Functional Disorders, Caused by Stress Factors

Anti-stress effect of the feed supplement composition (nutraceutical composition) was studied on layer replacement chickens, on broiler chickens and on layer and broiler breeders, in accordance to that disclosed previously in this document. Said nutraceutical composition in effective amounts was administered to chickens during second rearing period (50-120 days). Birds were weighed at the beginning of an experiment, in one week, and in one day before relocation, and at the moment of relocation (120 days). In accordance with these weighting results body mass loss, caused by moving, was calculated.

The nutraceutical composition as a stress reducing factor in industrial bird keeping conditions was also evaluated by feather pecking. The research was conducted on layer replacement chickens. From the 1^{st} (4^{th}) to 37^{th} days of life chickens received daily the nutraceutical composition in the form of AH in a dose of 300 mg per kg of live weight (10‰ of dry matter from total mass ration). Mortality rate was calculated daily, and the batches of poultry stock were inspected prior to moving for feather pecking cases and cull rates record. The results are shown in Table 22. Uniformity of live weight is evaluated by a variation coefficient (CV) in percents, wherein:

CV %=sd/mean of weight*100, wherein sd is a standard deviation.

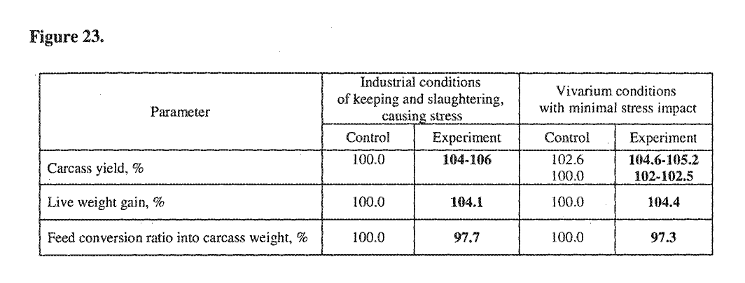

The nutraceutical composition in effective amounts was administered to broiler chickens, starting from 20 day of life until slaughtering (39-44 days). The most of broiler stock was kept in standard industrial conditions, wherein birds usually experience stress first during capture and then during transportation at the day of slaughtering. One experiment was carried out in conditions of vivarium, 21 days old broiler chickens were relocated whereto from the poultry-house. Anti-stress effect of the nutraceutical composition in this experiment was evaluated by changes in chicken's live weight during first few days after relocation to vivarium. Anti-stress effect of said composition at the end of the rearing period in broilers was determined by difference in slaughter weight of chickens, raised in industrial conditions and experiencing stress before the slaughtering, compared to that of chickens, raised and slaughtered in vivarium in conditions of minimum stress. Carcass yield was determined by calculating the mass ratio of eviscerated carcass to live weight prior to stress impact before slaughtering (table 23, FIG. 23).

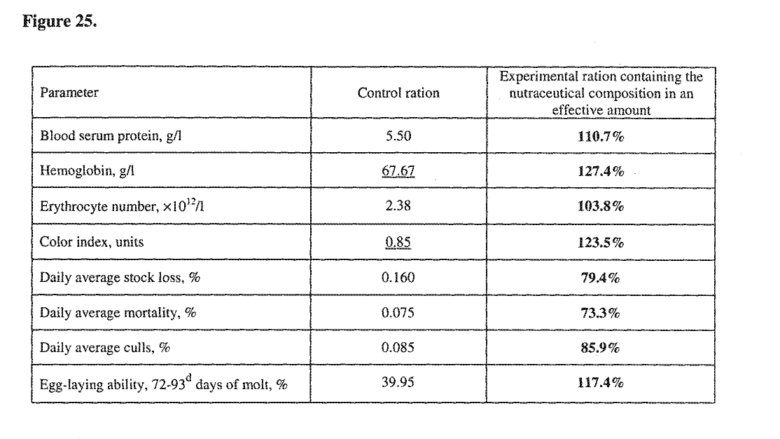

Said nutraceutical composition was administered to layer breeders by means of admixing it to feed at the end of a force molting period, while birds are 457-530 days old. Composition was thus administered starting from the 37^{th} day after the beginning of molt process, wherein a molt was caused by three-day starvation. Industrial and functional parameters, defining an adaptive potential in birds at the end of the force molt period, such as egg-laying ability, livability, body weight, hematological parameters and protein concentration in blood serum were determined. In 110 days after the beginning of starvation, causing, in turn, the molt process (age is 530 days), a control weighting was performed followed by a planned slaughtering of poultry stock, after that an average body mass was determined and carcass yield was calculated. In experimental and control batches blood parameters and histological texture of thyroid gland and spleen were studied (table 25, FIG. 25).

Said nutraceutical composition was administered to broiler breeders by means of admixing it to feed at the end of productive egg-laying period, starting from 322 days of life until the slaughtering at the age of 392 days. Prior to planned slaughtering a control weighing was performed, in order to determine an average live weight; and after the slaughtering an average body mass was determined and carcass yield was calculated.

According to the results thus obtained it was shown that in a control batch of layer replacement chickens, a live weight at the moment of moving was 12% lower than the estimated value, calculated with an allowance for a mass gain dynamics, that is indicative of weight loss by chickens in conditions of stress caused by capture and transportation. Body mass of those chickens that have received the nutraceutical composition corresponds to an expected value that is indicative of minimal weight loss in moving-caused stress conditions.

The experiment on three weeks old broilers, conducted after the relocation-caused stress, demonstrated, that during first three days after the relocation event 22.2% of chickens lost body weight, and all others possessed a decreased weight gain dynamics If in same conditions the nutraceutical composition would be included into chicken ration right after the relocation event, chickens would continue to gain weight without disturbance of weight gain dynamics. Feed consumption for one unit of body mass gain at this period in experimental batch was 3 times lower than in control.

It was shown that a carcass yield for broiler chickens of control batch that had experienced a minimal stress impact during slaughtering (in vivarium conditions) is 2.6% higher comparing to that of a control batch, slaughtered in industrial conditions (table 23, FIG. 23). A carcass yield for chickens that have received the nutraceutical composition was 4-6% higher in industrial conditions, but only 2-2.5% higher in vivarium conditions in comparison to that for the corresponding control batches (table 23, FIG. 23). Thereby, a positive effect of said nutraceutical composition on carcass yield is proportional to an intensity of an experienced stress impact that is indicative of an anti-stress nature of this effect. However, also in conditions of minimal stress impact, carcass yield for chickens, having said nutraceutical composition in their ration, was higher than that for control that may be indicative of other, than anti-stress, mechanisms, provided by this effect.

Results thus obtained indicate, that an increase in carcass yield as affected by said nutraceutical composition may be explained in terms of two mechanisms: an anti-stress impact of the nutraceutical composition that prevents a body mass loss during capture and transportation of poultry birds, and a metabolic effect, causing an increase of live weight in birds by building up the muscle and bone tissue. In vivarium conditions, wherein a preslaughtering stress impact on weight loss is brought to minimum, an increase in carcass yield, as affected by the nutraceutical composition, is mostly due to the metabolic effect. In industrial conditions, however, an increase in carcass yield, as affected by said nutraceutical composition, is due to both metabolic and anti-stress effects.

An existence of stress factor independent metabolic mechanism of action of said nutraceutical composition is confirmed by the fact, that the influence of said composition on live weight gain and on feed conversion rate in poultry is practically the same in industrial- and vivarium keeping conditions, in contrast to its influence on carcass yield (table 23, FIG. 23). In addition, said nutraceutical composition positively affects organoleptic and technological qualities of meat, causing improvement thereof. Thus, the fat content in meat increased by 17.6-22.3%, meat pH decreased by 16%, and acidity-oxidability coefficient decreased by 20% in comparison to that in control. These results are indicative of the improvement of functional state in chickens of experimental batches at the moment of slaughtering, of higher activity possessed by muscle enzymes, and of higher content of energetic substrates in muscle tissue. These differences between experimental and control chicken batches indicate, that adaptive potential of poultry organism gets improved under the influence of said nutraceutical composition, which makes poultry birds more prone to stress factors during capture, transportation and slaughtering.

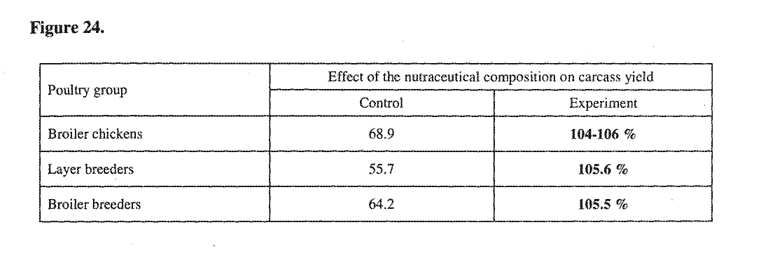

The results on carcass yield, obtained from kept in industrial conditions layer- and broiler breeders at the end of the egg-laying productive period were similar to that in broilers (table 24, FIG. 24). In experimental batches carcass yield increased 5.5-5.6% in comparison to that in control, which is indicative of an anti-stress effect of the nutraceutical composition in conditions of intensive stress impact on different groups of birds.

An experiment on layer breeders at the end of force molt period demonstrated, that even in 110 days after stress impact, caused by starvation and darkness, chickens in control batch had hemoglobin level and color index below normal, that is characteristic of a condition of hypochromic anemia (table 25, FIG. 25). Upon administration of the nutraceutical composition hemoglobin level and erythrocyte number are increased, and a color index is restored back to normal, that is indicative of hematopoiesis recovery. In addition, said composition promotes an increase of protein concentration in blood serum. The study of industrial parameters in poultry batches, the nutraceutical composition was administered thereto, had demonstrated a reduced stock loss, herein, reduced mortality and culls rates, as well as improved egg-laying ability (table 25, FIG. 25).

On the histological level, changes in spleen texture were found, indicative of excessive functional load on said organ related to an increase in egg production after completion of molt period. In those egg-laying hens, which have received the nutraceutical composition, these changes were less pronounced.

In thyroid gland texture the morphological signs of exhaustion were found, wherein exhaustion is caused by an excessive functional activity of said gland during the period of completion of force molt. In those layers, which have received the nutraceutical composition, histopathologic features of thyroid gland corresponded to normal. Those results provide a direct experimental evidence for one of the anti-stress effect mechanisms said nutraceutical composition, which mechanism lies in rising the compensatory potential of endocrine system in conditions of recovery after starvation- and darkness caused stress impacts.

The results obtained therefore are indicative of that said nutraceutical composition possesses an anti-stress effect, thus preventing live weight loss in conditions of stress impact during capture, transportation and slaughtering of poultry birds, which results in improved meat quality. Similarly said composition provides a protection from multiple stress factors of internal and external environment, such as nutritional, temperature or light factors, which inevitably affect poultry in conditions of intensive industrial farming. The possible mechanism of anti-stress action of said composition lies in an improvement of the adaptive potential of nervous and endocrine systems thus promoting a production of adaptive energy in an organism.

### Example 12

### Improvement in functional stability of cells and tissues in regards to damaging effects of oxidative stress factors.

In conditions of industrial poultry keeping various functional systems experience an excessive oxidative load, resulting in a range of functional dysfunctions and in variety of disorders. In order to reduce damaging effects of external environment, otherwise leading to oxidative stress, and in order to improve functional stability of cells and tissues, natural and synthetic antioxidants are widely utilized, being normally admixed to feed.

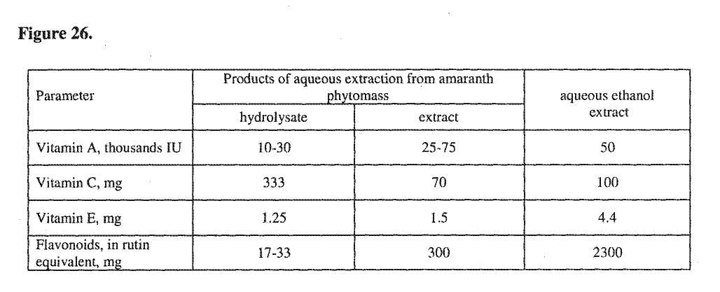

The feed supplement composition (nutraceutical composition), as determined by analytical methods, comprises the most effective phytogenic antioxidants, such as vitamins A, C, E and flavonoids. Results of the analytical study are shown in Table 26 of FIG. 26.

Oral administration of said nutraceutical composition in effective amounts ensures an improvement in functional stability (antioxidative protection) of cells and tissues in regards to damaging effects of oxidative stress factors.

### Example 13

### Preventive Measures Against Infectious Diseases Alternate to Prophylactic Drugs, Including Broad Spectrum Antibiotics and Vitamins

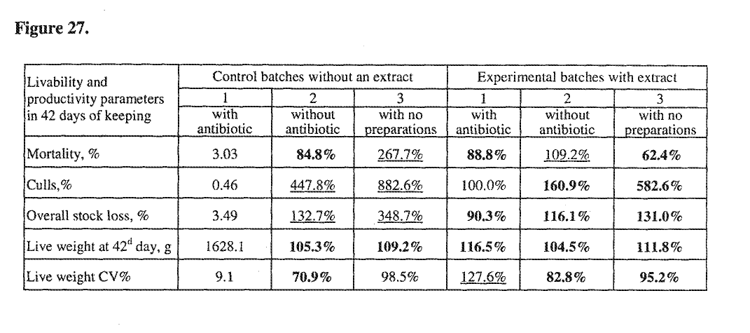

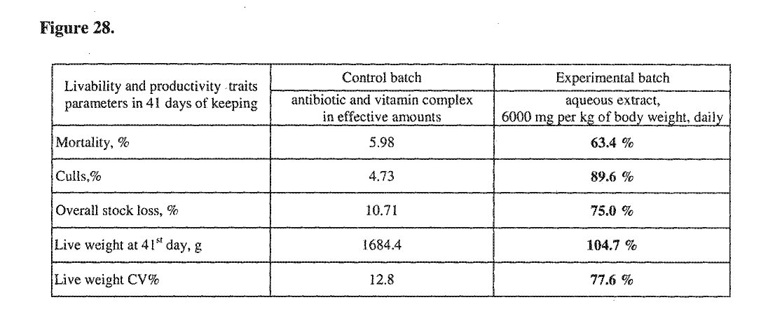

Herein, three experiments on broiler chickens were conducted. In the first two experiments a prophylactic action of the feed supplement composition (nutraceutical composition) in the form of aqueous extract was compared to that of known veterinary preparations, such as an antibiotic Colmik, vitamin complex Introvit and glucose, in various combinations. Said veterinary preparations were administered in the following combinations: all three at once; two preparations without antibiotic; two preparations without glucose; and just water. Chickens in control batches during first three days of life have received either water or said veterinary preparations in effective amounts in a form of drinking solution. In experimental batches same preparations were either diluted in the aqueous extract, or said aqueous extract was administered without addition of preparations. An average dosage of the aqueous extract is 20-25 ml, provided that dry content comprises 1.5-2% that corresponds to 0.4-0.5 g of dry extractive substance per head daily, or 6000 mg per kg of live weight. An aggregate consumption of the aqueous extract in three days comprised 18000 mg per kg of live weight. Chicken flock in each batch was 1750 head in the first experiment and 3276 head in the second experiment. Experimental results are shown in tables 27 and 28 of FIGS. 27 and 28, respectively.

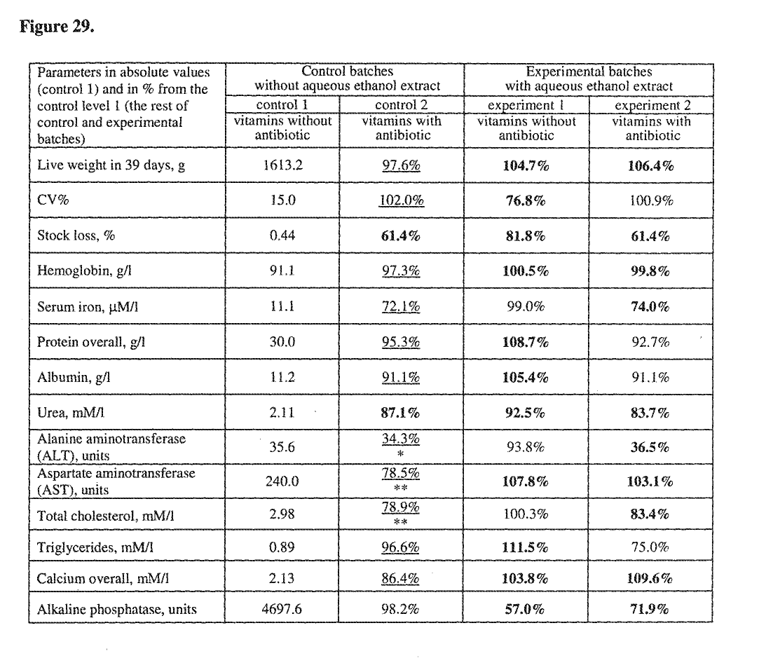

In the third experiment a prophylactic action of an antibiotic was compared to that of the nutraceutical composition in the form of aqueous ethanol extract (AEE) against the background of a standard vitamin complex. An aqueous solution of Doxy antibiotic and vitamin complex Rexvital in effective amounts was administered to chickens from the control batch during first three days of life. Chickens from the experimental batch have received during the same period only AEE with 1000-fold dilution. Daily AEE consumption per head, calculated by dry content, comprised 11.2 mg. Average daily dosage of AEE, calculated by its dry content per kg of live chicken weight, comprised 200 mg/kg, and an aggregate consumption in three days comprised 600 mg/kg. Each batch comprised 350 head. Mortality and cull rates were registered daily in all experiments; and control weighting was conducted weekly. Experimental results are shown in Table 29 of FIG. 29.

In accordance with the results obtained it seems, that the administration of the nutraceutical composition in the form of aqueous extract (AE) as a drinking solution jointly with all three prophylactic veterinary preparations causes the reduction in stock loss by 9.7% and an increase in chicken's average live weight at the end of rearing period by 10.8% (exp. 1, table 27, FIG. 27).

Upon antibiotic withdrawal, but further use of glucose and vitamin complex, an overall stock loss in control batch increased almost 33% (control 2, table 27, FIG. 27), however in corresponding experimental batch it increased only by 16% (exp. 2, table 27, FIG. 27).

Upon withdrawal of all three prophylactic veterinary preparations in control batch (control 3, table 27, FIG. 27) mortality rate increased 2.7-fold, cull rate- 8.8-fold, and an overall poultry stock loss—3.5 fold. In the experimental batch with no preparations (exp. 3, same table and figure) mortality rate was lower not only in comparison with the corresponding control batch 3, but also in comparison with the control batch that have received an antibiotic (control 1, FIG. 27); however, an amount of culls decreased only in comparison to the corresponding control batch, remaining 5.8 times higher than that in the control with antibiotics. An overall poultry stock loss in the experimental batch 3 increased by 31% in comparison with the control batch 1, however decreased 2.7-fold in comparison with the corresponding control batch 3 (table 27, FIG. 27).

Livability rate for the control batch, the nutraceutical composition was administered whereto instead traditional prophylactic preparations (exp.3, FIG. 27), stands closest to the control batch received no antibiotics, but vitamin complex and glucose (control 2, FIG. 27). This implies, that said nutraceutical composition, administered as prophylactic measures in the form of drinking solution during the initial period of broiler chicken keeping, effectively substitutes those complex preparations intended to provide auxiliary prophylactic effects by means of vitamin- and energy-providing components they comprise, whereas an antimicrobial effect of antibiotics mostly reduces the risk of infectious diseases, otherwise causing loss of chicken stock. In case of joint administration of said nutraceutical composition and the prophylactic complex preparation comprising vitamin- and energy-providing components (exp.2, FIG. 27), the synergetic effect is provided, which leads to almost three-fold decrease in an amount of culls and, therefore, already being ill chickens in comparison to that in control batch 2, no nutraceutical composition was administered whereto. The term “prophylactic complex preparation comprising vitamin- and energy-providing components”, herein, refers to vitamin complex and glucose administered jointly.

In the control batch that received no prophylactic preparations (control 3, table 27, FIG. 27) live weight of chickens at the end of rearing period was higher, than that in control with an antibiotic (control 1, FIG. 27). This implies that an administration of antibiotic as prophylactic measures causes weight gain reduction in broilers, in other words has a negative effect on their productivity (FIG. 27). Nutraceutical composition thus eliminated the negative effect an antibiotic has on weight gain, acting more effectively in presence of said antibiotic (exp. 1, FIG. 27), than per se (exp. 3, FIG. 27). Again, this effect may be explained only by synergetic interaction of the nutraceutical composition with prophylactic complex preparations comprising vitamin- and energy-providing components.

In the second experiment (table 28, FIG. 28) it was shown that an average live weight of chickens in experimental batch, the nutraceutical composition in the form of aqueous extract was administered whereto during the first three days of life, instead of Colmik antibiotic and vitamin complex Introvit, was 4.7% higher and CV % lower in comparison with that in control batch; and mortality rate was 1.6 times lower, correspondingly. Cull rate in experimental batch decreased by 10.4% and an overall poultry stock loss—by 25% (table 28, FIG. 28).

Results obtained in the second experiment, wherein the complex of prophylactic preparations was completely replaced by the nutraceutical composition in the form of aqueous extract (table 27, FIG. 27), differ substantially from the results obtained in analogous conditions in the first experiment (exp.3, table 27, FIG. 27), wherein an overall poultry stock loss parameter in the experimental batch with nutraceutical composition had increased because of the abundance of culls in comparison with the same parameter for control batch, a complete complex of prophylactic preparations was administered whereto (control 1, table 27, FIG. 27). These differences may be explained by diverse epizootic situation in experiments compared.

In the first experiment, in a satisfactory epizootic environment, prophylactic administration of antibiotic jointly with other preventive preparations provides sufficiently low poultry stock loss rate (3.49%) and negligible cull rate (0.46%).

In the second experiment an intensity of infections in broiler chickens population was sufficiently higher, for which reason the standard complex of prophylactic preparations could not provide required efficiency rates anymore. For example, in comparison with the first experiment herein mortality rate was 2 times higher, cull rate—10 times higher, and an overall poultry stock loss—3 times higher. Upon such a significant infectious load, a complete replacement of the standard complex of prophylactic preparations by the nutraceutical composition provides a substantial improvement in poultry stock preservation rates, not only by reducing mortality rates (reducing the probability of fatal outcome), occurring also in more favorable epizootic environment in the first experiment, but also by reducing an amount of culls (recovery of some part of ill chickens).

In the third experiment (table 29, FIG. 29) it was shown that administration of antibiotic as a part of a prophylactic preparation complex led to decrease in average body mass and uniformity of live weight of broiler chickens at the end of rearing period and caused a range of disturbances in hematological and biochemical blood parameters (control 2) as compared with the control batch without an antibiotic (control 1). Prophylactic administration of said nutraceutical composition in the form of aqueous ethanol extract jointly with an antibiotic led to increase in average body mass in broiler chickens by 6.4%, and in an absence of the antibiotic—by 4.7%. Meanwhile an improvement in chicken's functional state was registered in both experimental batches as compared to control 1; moreover it seems that in the batch, the nutraceutical composition jointly with an antibiotic is administered whereto, the severity of caused by said antibiotic disturbances decreases (table 29, FIG. 29).

Dysfunctions in hematopoiesis were also registered upon administration of antibiotic, which expressed in reduction of both hemoglobin levels and iron concentration in blood serum (control 2). In case of prophylactic use of the nutraceutical composition in the form of aqueous ethanol extract in chickens during the first five days of life, without an antibiotic (exp.1), both hemoglobin and blood serum iron levels slightly differ from that in the control 1. The administration of said aqueous ethanol extract together with antibiotic (exp. 2) reduced the suppressing effect of antibiotic on hematopoiesis process (table 29, FIG. 29).

Dysfunctions in protein metabolism had occurred under the influence of antibiotic, thus expressed in reduced overall protein level and albumin fraction in blood serum and in reduced ALT and AST transaminase enzyme's activity, which enzymes play important role in protein synthesis in the organism. On the contrary, upon utilization of the nutraceutical composition instead of antibiotic, overall protein- and albumin fraction levels in blood serum rise. AST activity in both experimental batches increased moderately, in limits of physiological standard. ALT activity was moderately reduced in the experimental batch without antibiotic, remaining in limits of physiological standard (exp. 1, FIG. 29). Administration of the nutraceutical composition jointly with antibiotic led to partial restoration of ALT activity (exp. 2, FIG. 29). These results indicate that utilization of said nutraceutical composition as a preventive measure, alternate to antibiotics, promotes enhancement in protein metabolism in broilers; and utilization of said composition jointly with antibiotic reduces metabolic disturbances otherwise originating under the influence of antibiotic.

Overall cholesterol and triglyceride level parameters, reduced in control 1, were reduced even more in control 2, which is indicative of the nutritional supplies decrease in an organism caused by dysfunctions in lipid metabolism. Joint administration of said nutraceutical composition with antibiotic did not lead to the elimination of said metabolic dysfunctions, caused by antibiotic. Upon administration of the nutraceutical composition instead of antibiotic cholesterol concentration remained at the same level; however the level of triglycerides in blood serum had increased, which is indicative of partial recovery of lipid metabolis functions as affected by said nutraceutical composition.

In the course of same experiment the comparative effects of the nutraceutical composition and an antibiotic on the parameters of mineral metabolism were studied (table 29, FIG. 29). In the control batch without an antibiotic an alkaline phosphatase activity was several times above normal, which is a sign of calcium deficiency in the organism, despite of normal calcium levels in blood (control 1). Administration of antibiotic only enhanced the disturbances in mineral metabolism, which was expressed in reduced calcium levels in blood, but an increased activity of alkaline phosphatase (control 2). Upon administration of the nutraceutical composition the parameters for mineral metabolism were back to norm, which was expressed in increased calcium levels and reduced activity of alkaline phosphatase in both experimental batches, with or without antibiotic.

Chicken livability in the third experiment was exceptionally high in all poultry batches, an overall stock loss even in the control batch without antibiotic comprised only 0.44% (table 29, FIG. 29). This may be a consequence of high-grade sanitary arrangements during incubation, sorting and moving of one day old chicks herd in order to conduct this industrial experiment. In conditions of so low infectious load on broiler chicken herd an administration of said nutraceutical composition jointly with antibiotic (exp. 2) did not lead to additional improvements in chicken livability as compared to the control, wherein an antibiotic was utilized (control 2). In case the nutraceutical composition in the form of aqueous ethanol extract was used instead of the antibiotic (exp. 1), then its prophylactic effect on the livability was only slightly inferior to that of the complete set of traditional prophylactic preparations, including antibiotics (control 2).

Utilization of said nutraceutical composition instead antibiotics as a preventive measure, which reduces the risk of infectious diseases and increases the resistance of poultry birds to negative consequences of such diseases, would depend on the level of infectious load in poultry population. In conditions of favorable and satisfactory epizootic environment said nutraceutical composition has a positive effect on poultry functional state, providing an improvement in productivity upon slight reduction in livability in comparison to poultry batches that were administered with antibiotic. Said nutraceutical composition, however, does not reduce the risk of infectious diseases, but significantly increases an overall resistance in poultry, thus substantially facilitating the course of a disease and reducing the danger of lethal outcome. In conditions of unfavorable epizootic environment, when antibiotics do not possess the desired effects of reducing risks of infectious diseases anymore, said nutraceutical composition may act more effectively, comparing to antibiotics, providing higher livability rates. Thus, said nutraceutical composition may be used as more effective and safe preventive means, as compared to antimicrobial preparations of broad spectrum.

### Example 14

### Digestion Enhancement Upon Administration of the Nutraceutical Composition as a Growth-Promoting Phytobiotic Alternate to In-Feed Antibiotics.

During the experiments on broiler chickens, the ration of said chickens contained, in addition to basic formula, either in-feed growth-promoting antibiotics Salinomycin and Zink-bacitracin, or a probiotic feed supplement Cellobacterin, or a phytogenic feed supplement in the form of an aqueous hydrolysate. As mentioned above, the nutraceutical composition may be used either as phytogenic feed supplement or as veterinary means. Herein, the terms “phytogenic feed supplement” and “phytobiotic” will be used to denote a feed supplement of a plant origin, herein, originated from amaranth. The term “probiotic”, herein, denotes a feed supplement worked out on the basis of live microorganisms beneficial to host organism. All preparations were administered in effective amounts. For the evaluation of an efficiency of phytobiotic in comparison to growth-promoting antibiotics, or, against the background of said antibiotics, said phytobiotic in the form of an aqueous extract was admixed to feed formula in an amount of 2‰ by dry weight.

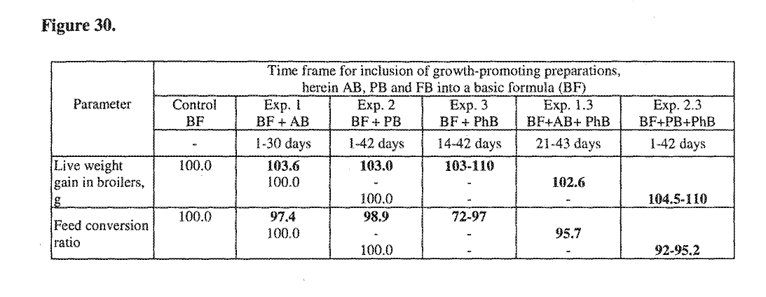

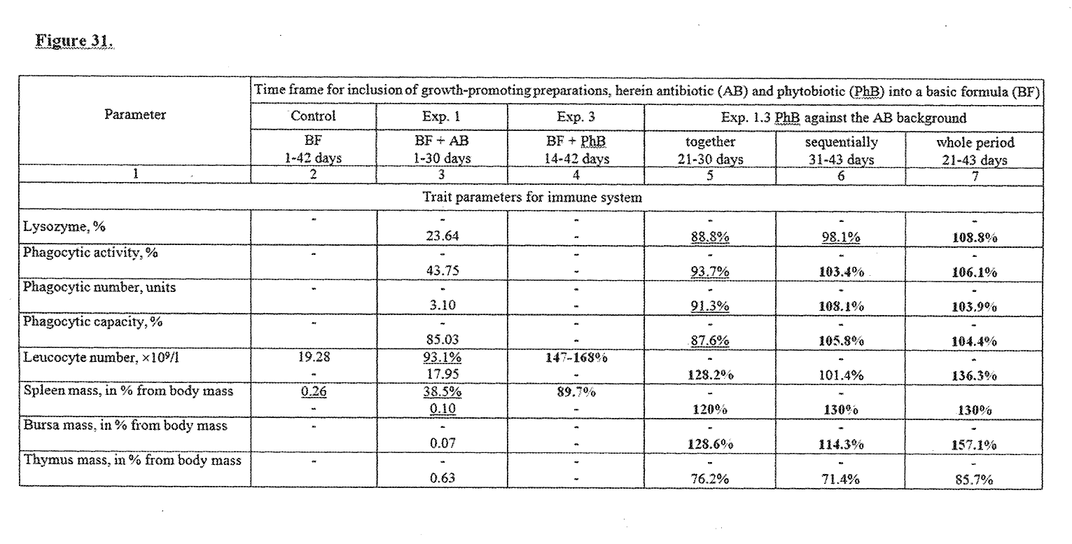

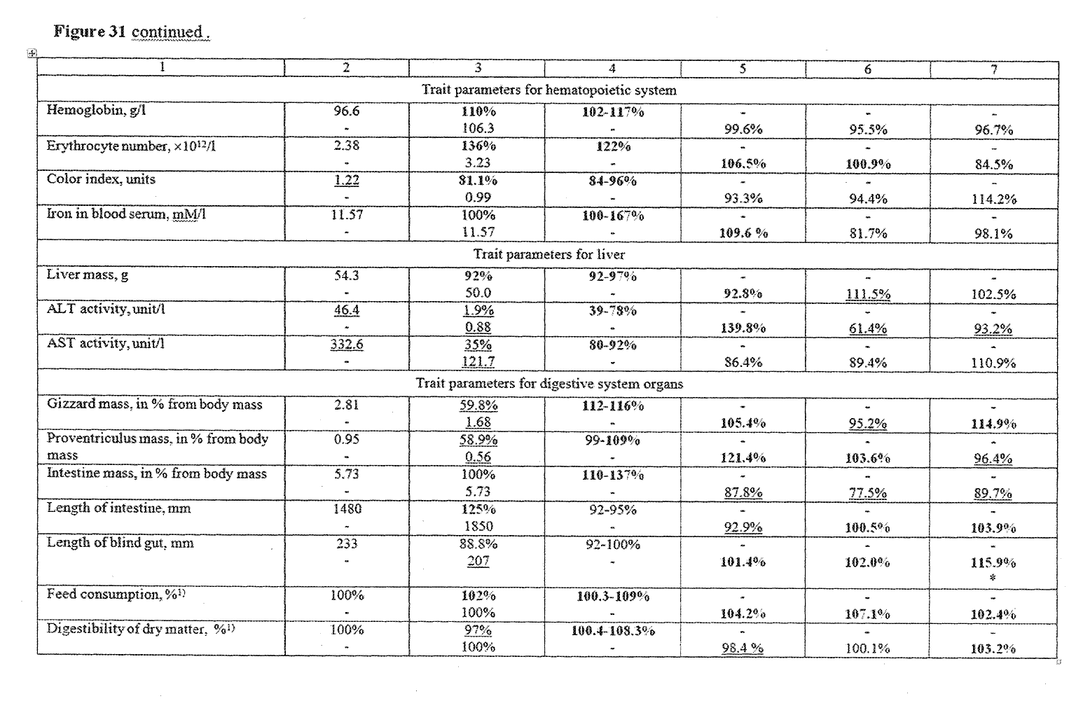

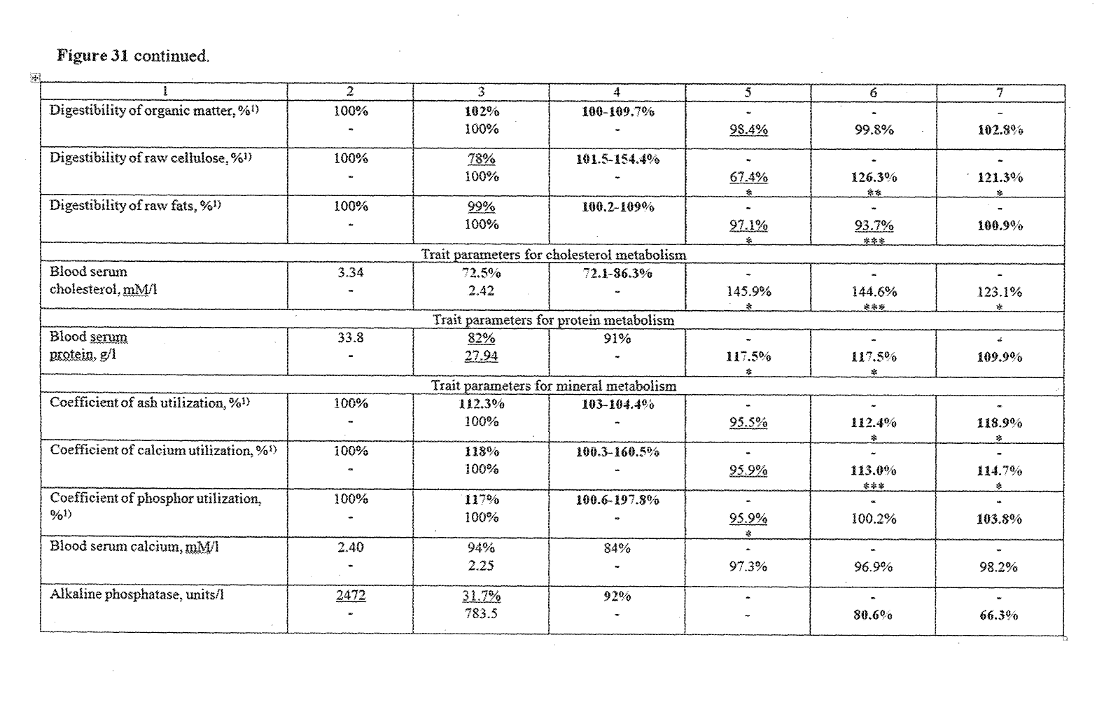

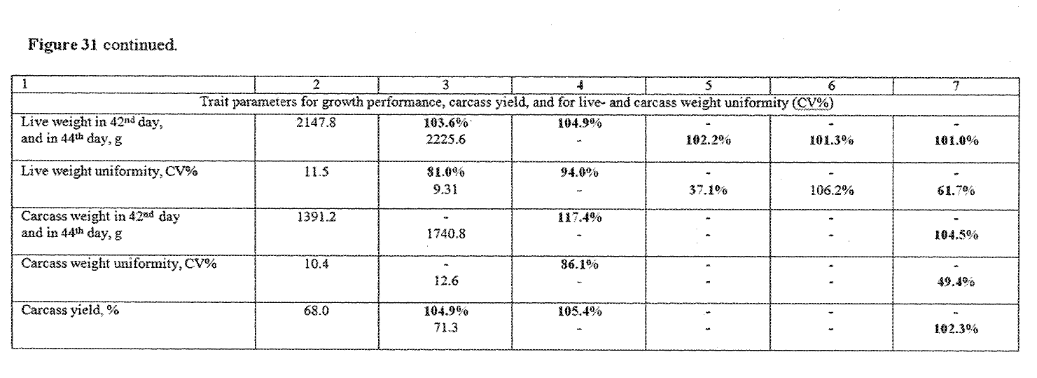

It was shown that the nutraceutical composition as a phytogenic feed supplement improves live weight gain and feed conversion rates in broilers more efficiently in comparison to growth-promoting antibiotics and probiotics (exp. 1-3, tables 30, 31, FIGS. 30, 31, respectively). Utilization of said composition as an alternative to growth-promoting antibiotics leads to the improvement in poultry productivity and enables obtaining safe, in regards to human health, organic poultry products (exp. 3, table 30, FIG. 30). Utilization of said composition leads to an increase in average carcass weight and carcass weight uniformity (table 31, FIG. 31).

It is also of interest to compare the effects said nutraceutical composition and growthpromoting antibiotics have on a functional state of poultry. Growth-promoting effect of infeed antibiotics is provided by their positive influence onto several functional systems in poultry. Thus, in-feed antibiotics improve hematopoietic functions, reduce liver hypertrophy, reduce cholesterol level in blood serum, increase the length of an intestinal canal, and improve feed consumption, digestibility of dry matter and intensity of mineral metabolism, including utilization of ash, calcium and phosphor contained in fodder (exp.1, table 31, FIG. 31).

However, along with their positive effect on some functional systems, growth-promoting antibiotics cause malfunctioning of the other systems of an organism (exp.1, table 31, FIG. 31). Thus, in-feed antibiotics cause significant decrease of spleen mass, dramatic reduction in alkaline phosphatase, ALT and AST enzyme's activity, decrease of both proven-triculus and gizzards mass, reduced digestibility rate of basic nutritional components of the ration, and blood serum protein level reduction. Although inducing a productivity gain by activation of certain functional systems, in-feed antibiotics suppress the other functional systems, causing impairments in poultry functional condition and thus limiting the possibilities to productivity improvement. In addition, in-feed antibiotics for poultry cause a rise in antibiotic resistance level in human populations, consuming the meat of said poultry.

Administration of the nutraceutical composition as an alternative to in-feed antibiotics not only promotes an additional productivity gain in broiler chickens (exp. 3, table 30, FIG. 30), but also has a positive effect on all functional systems of said chickens (exp. 3, table 31, FIG. 31). Administration of said nutraceutical composition promotes an increase in hemoglobin concentration, rise in erythrocyte and leucocyte number and serum iron levels, increase in gizzard and proventriculus mass, as well as in intestine mass, and improvement of feed consumption rates and of digestibility parameters for all nutritional components of the ration. At the same time a hyperplasia reduction in liver and spleen is recorded, as well as a reduction in ALT and AST enzyme's activity and a decrease in cholesterol levels in blood serum.

The nutraceutical composition, being administered against the in-feed antibiotics background, preserves its stimulating effect on productivity and feed conversion rates (exp.1.3, table 30, FIG. 30), on immune and digestive systems and on trait parameters of protein and mineral metabolism (exp.1.3, table 31, FIG. 31).

However, when being administered jointly with in-feed antibiotics, the effect of said nutraceutical composition on some functional systems becomes less pronounced, or disappears completely (exp. 1.3, table 31, FIG. 31). In particular, severe impairments of immune and digestive systems, as well as in mineral metabolism in broilers were observed after short term administration of phytobiotic composition (at the age of 21-30 days) jointly with antibiotic. Also in same batch of chickens no effect of said phytobiotic composition on live weight gain was developed, however, feed consumption on weight gain unit increased by 2%. These data are indicative of that, joint administration of the nutraceutical composition and growth-promoting antibiotics may lead to downregulation of certain functional systems, in case of exceeding the compensatory resources of regulatory systems.

### Example 15

### Method for the Improvement of Poultry Livability

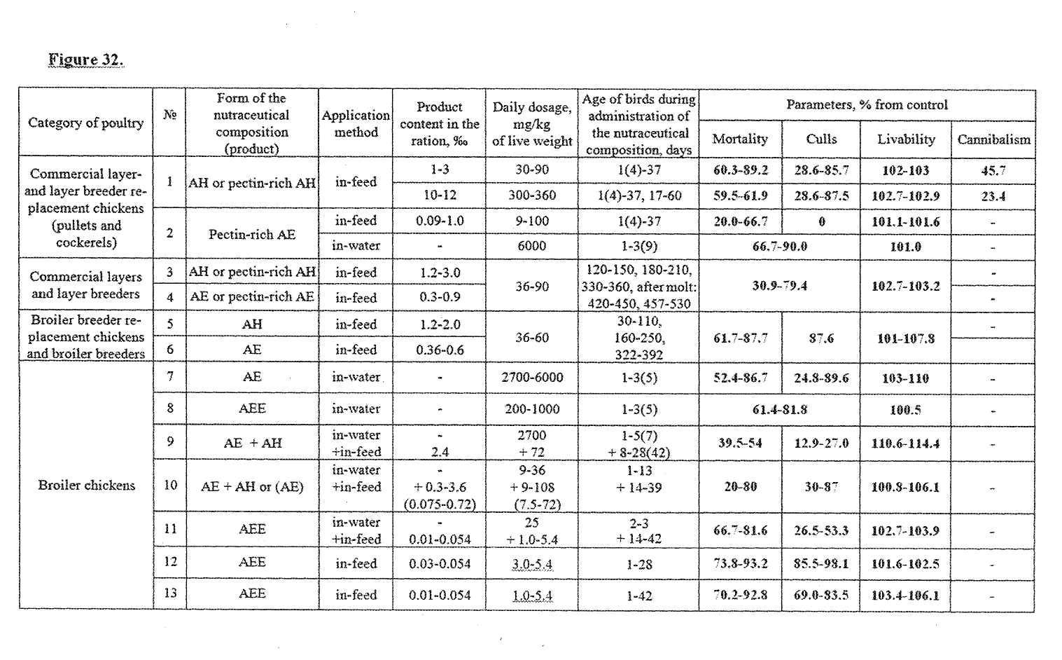

In accordance to that disclosed previously in this document, the feed supplement composition (nutraceutical composition) was administered to poultry feed formula in effective amounts. The poultry groups, herein, comprised layer replacement chickens, broiler chickens, layer breeders and broiler breeders. The effect of said nutraceutical composition on livability parameters was determined by daily account for stock loss (fatal cases) and for culls (weak bird's rejection), followed by stock re-count with respect to reference number originally taken into an experiment. The results are shown in Table 32 of FIG. 32.

As affected by said nutraceutical composition, a significant reduction in stock loss and in cull rates was observed. Administration of said composition at different time periods led to an increase in livability rate for layer and broiler breeders by 0.5-14.4% (table 32, FIG. 32).

### Example 16

### Methods to Improve Feed Consumption Rates and to Increase an Assimilability of Nutritional Compounds of the Ration

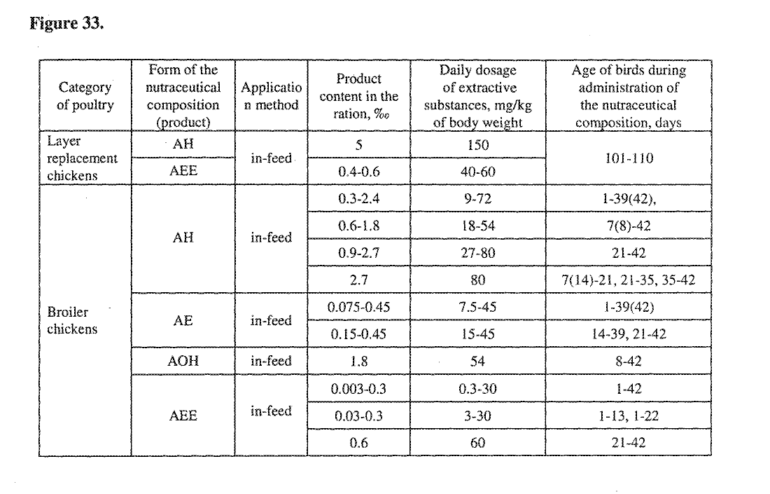

In accordance to that disclosed previously in this document, the feed supplement composition (nutraceutical composition) was administered to poultry feed formula in effective amounts (table 33, FIG. 33). The poultry groups, herein, comprised layer breeder replacement chickens and broiler chickens.

As a result of administration of said composition, feed consumption rate had increased by 3.0-13.4%, which is indicative of the improvement of organoleptic properties of feed, causing in turn an increase in feed palatability for poultry. At the same time an increase in feed assimilability was observed, based on evaluation of digestibility coefficients for main nutritional components of the ration.

A digestibility coefficient for nutritional component, the degree of its increase (%) and the significance level of probability (p) for the effect obtained may be expressed the following way:

| | | |
|---|---|---|
| for dry matter | 1.4-8.3%; | p < 0.001; |
| for organic matter | 1.5-9.7%; | p < 0.001; |
| for raw cellulose | 4.0-54.4%; | p < 0.001; |
| for raw fats | 3.5-9.0%; | p < 0.001; |
| for nitrogen-free extractive compounds | 3.0-6.7%; | p < 0.001; |
| for raw protein | 0.4-5.3%; | p < 0.001. |

### Example 17

### Method for Promoting Weight Gain in Replacement Chickens, and for Improving Growth Performance, Carcass Yield and Meat Quality in Broiler Chickens.

In accordance to that disclosed previously in this document, the feed supplement composition (nutraceutical composition) was administered to poultry feed formula in effective amounts. The poultry groups, herein, comprised commercial layer- and layer breeder replacement chickens and broiler chickens.

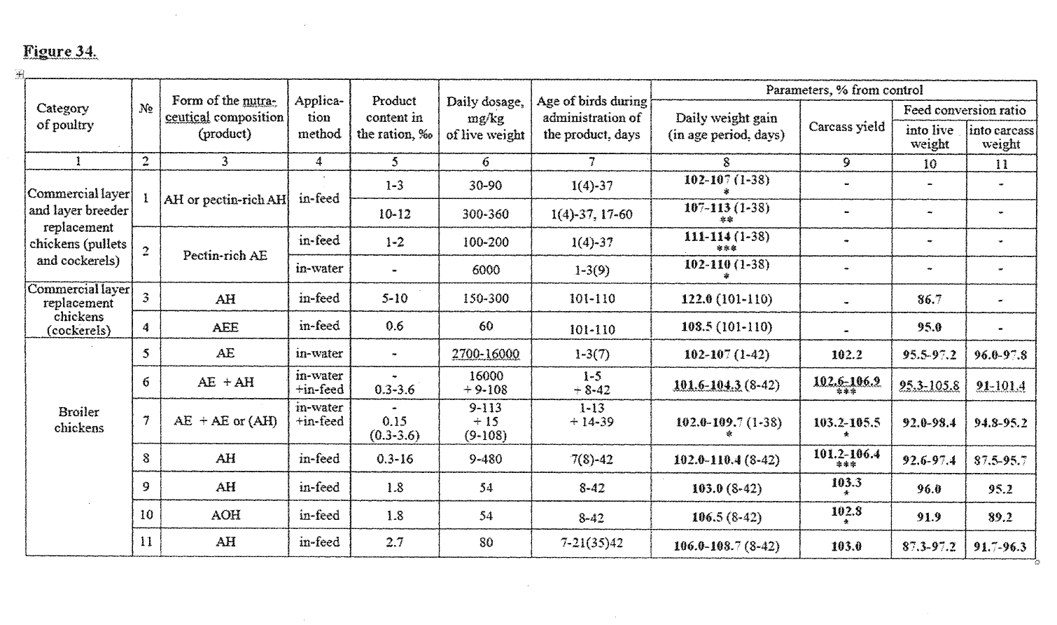

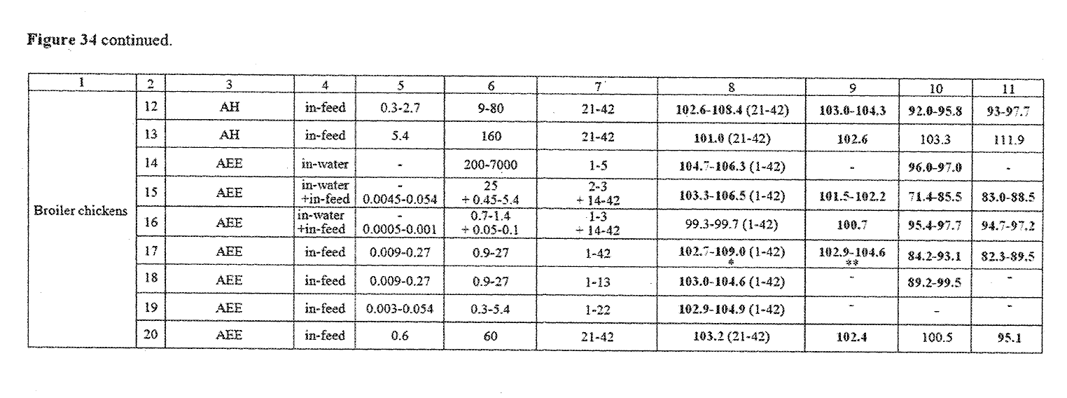

An intensity of weight gain in chickens was estimated by results of control weightings, by determination of an average weight of chickens at the moment of weighting and of a weight gain over a time period between weightings. Feed consumption was measured every day. Carcass yield for broilers was calculated as a mass ratio of eviscerated carcass to live weight of chicken before slaughtering. Feed conversion was calculated per live weight gain unit and per meat production unit. Product quality was evaluated by biochemical studies and by tasting of boiled meat and broth by the group of experts. Results are shown in tables 34-37 of FIGS. 34-37, respectively.

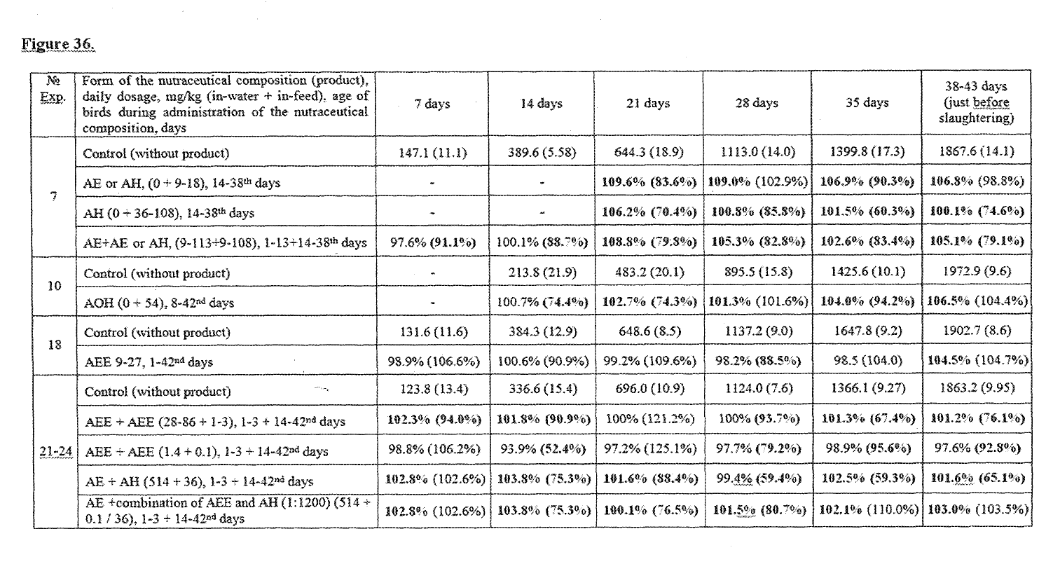

Administration of said nutraceutical composition to layer replacement chickens at different age periods during rearing thereof promotes increase in daily weight gain in pullets by 7-14% and in cockerels by 8.5-22%. Administration of said composition to broiler chickens in effective amounts during the whole rearing period promotes an increase in live weight gain by 9.0-10.4% (table 34, fig.34). In most of experiments conducted at different age periods it was found that live weight and flock uniformity parameters in experimental batches of broiler chickens was higher in comparison with corresponding parameters in control batches (table 36, FIG. 36). For achieving similar results with short term administration of said composition, the dosage thereof must be increased essentially.

The nutraceutical composition also provides an increase in carcass yield for broilers by 4.6-5.5% in comparison to control. Increase in poultry productivity is attained by substantial improvement of feed conversion rates. Thus, feed conversion per kilogram of live weight gain in replacement chickens is decreased by 5.0-13.3%; similarly, feed saving per kilogram of live weight gain in broiler chickens may reach 24.1-28.6%. At the same time the decrease in feed consumption for obtaining a kilogram of meat product may reach 17.7% (table 34, FIG. 34).

A performance comparison between various forms of the nutraceutical composition, as products of aqueous, aqueous-oil and aqueous-ethanol extraction, in regards to meat productivity of broilers and feed conversion (table 34, FIG. 34) shows, that same effect may be achieved upon utilization of substantially lesser amounts of an extractive substance, obtained by means of aqueous-ethanol extraction of amaranth phytomass. Thus, a minimum effective dose for the extractive substance in aqueous ethanol extract in feed formula comprises only 0.003‰ (3 g per ton), whereas for products of aqueous extraction this dose is 50-100-fold higher, 0.15‰ for extracts and 0.3‰ for hydrolysates, correspondingly.

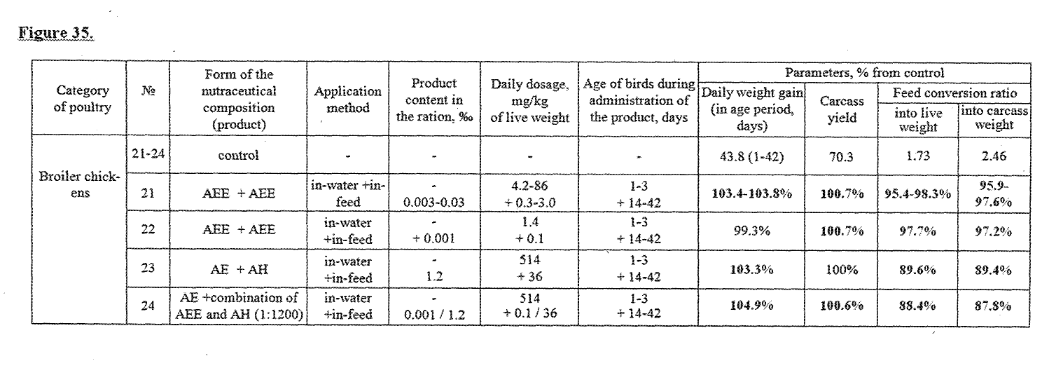

Thus, it is economically reasonable to utilize an aqueous ethanol extract as means for improving qualities of basic feed supplement in the form of aqueous hydrolysate. Meanwhile, an increase of effectiveness of utilization of said aqueous hydrolysate is reached by using the aqueous ethanol extract in very low dosage (0.001‰), which dosage in not effective when said aqueous ethanol extract is administered alone (table 35, FIG. 35).

During growth dynamics analysis in chickens it was shown, that the nutraceutical composition promotes an increase in live weight at different age periods and an improvement in live weight uniformity (table 36, FIG. 36), the evidence thereof is provided by the decrease in variation coefficients (CV %) in experimental batches in comparison to that of control.

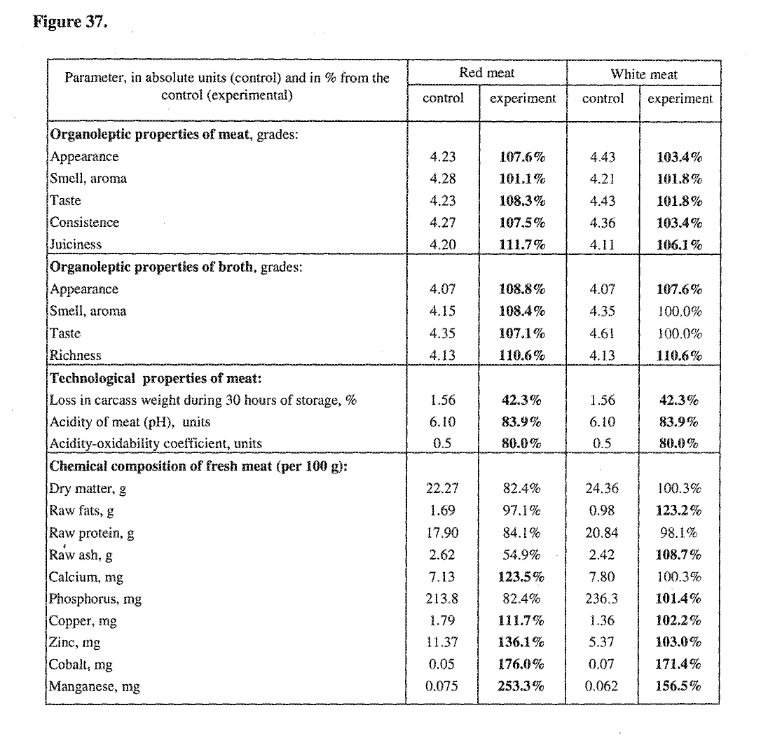

Along with increasing broiler's productivity, administration of the nutraceutical composition may lead to quality improvement of products obtained, expressed in improved organoleptic, biochemical and technological parameters of meat (table 37, FIG. 37).

In experimental batches the nutraceutical composition was administered thereto almost all tasting evaluation grades for meat and broth organoleptic parameters have improved. Loss in carcass weight throughout 30 hours of refrigerated storage, specified by technological regulations of meat ripening, decreased by 41-48%, which is indicative of an improvement of technological properties of meat, in particular, of an increase of its moisture-keeping ability. Acidity of meat (pH) decreased by 2-16% and acidity-oxidability coefficient decreased by 20%, that is indicative of more effective meat ripening process upon its refrigerated storage, specified by the technology and intended to improve the product's flavor (table 37, FIG. 37).

In white meat an increased content of fats, ash, calcium and phosphorus were recorded, and in red meat an increased moisture load was recorded, that is indicative of an improved moisture-keeping ability and higher juiciness. In both white and red meat the content of microelements, such as copper, zinc, cobalt and manganese, increases (table 37, FIG. 37). These results are indicative of an improvement of biological value of meat, obtained from poultry raised with the utilization of said nutraceutical composition.

### Example 18

### Method for the improvement of egg-laying productivity in commercial layers and breeding capacity in layer and broiler breeders.

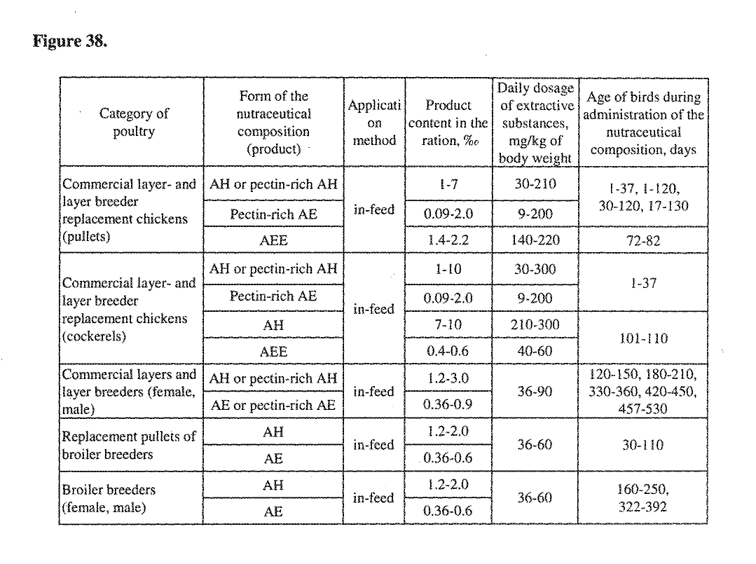

In accordance to that disclosed previously in this document, the feed supplement composition (nutraceutical composition) was administered to poultry feed formula in effective amounts (table 38, FIG. 38). The poultry groups, herein, comprised replacement chickens in different rearing periods, commercial layers, layer- and broiler breeders in different egg-laying periods. As a result of administration of said composition, improvements in egg-laying productivity and breeding capacity of above mentioned poultry groups were recorded.

Egg-productivity parameters for commercial layers and layer breeders provided with the rations, comprising the nutraceutical composition, degree of variability thereof (in % from control), and the significance level of probability (p) for the effect obtained may be expressed the following way:

| | | |
|---|---|---|
| Increase of egg-laying ability during basic egg- | 4-16%; | p < 0.05; |
| laying cycle | | |
| Increase of egg-laying ability during critical | 18-84%; | p < 0.05; |
| periods of egg-laying cycle | | |
| Increase in time length of peak period of egg- | 7-10 days; | |
| laying ability | | |
| Increase in time length of egg-laying period | 12-17 days; | |
| Increase in average egg weight | 2.0-6.2%; | p < 0.05; |
| Decrease in feed amount used for every 10 eggs | 5.3-11.3%; | p < 0.05; |
| Decrease in feed amount used for kg of egg- | 5.6-11.9%; | p < 0.05. |
| production | | |

Breeding capacity parameters for layer and broiler breeders on the diets, comprising the nutraceutical composition, degree of variability thereof (in % from control), and the significance level of probability (p) for the effect obtained may be expressed the following way:

| | | |
|---|---|---|
| Increase in average weight of an settable egg | 2.0-6.2%; | p < 0.05; |
| Increase in settable eggs yield | 3-9%; | |
| Decrease in amount of non-fertilized eggs | 6.0-14.4%; | p < 0.05; |
| during basic egg-laying cycle | | |
| Decrease in amount of non-fertilized eggs in | 25.1%; | p < 0.05; |
| the beginning of the egg-laying period | | |
| Increase in hatchability rates of settable eggs | 2.0-6.5%; | p < 0.05; |
| during basic egg-laying cycle | | |
| Increase in hatchability rates of settable eggs | 28.4%; | p < 0.05; |
| in the beginning of the egg-laying period | | |
| Increase in amount of hatched chicks | 14%; | |
| per every 1000 of hens of a breeder flock | | |
| Increase in average weight of one day old | 2%; | p < 0.05. |
| chicks | | |

1. A method for feeding poultry comprising:
(a) obtaining a feed supplement composition comprising an aqueous ethanol extract isolated from leafy parts of Amaranthus hybridus by using a mixture of water and ethanol as an extractive agent, in which mixture an amount of ethanol is within a range of 0.1-85 vol-%,
(b) admixing said composition into a basal poultry feed or drinking water in an amount of at least 0.003 g/kg, and
(c) administering the resulted mixture to poultry in accordance with predetermined dosage regimen.

2. The method of claim 1, wherein the feed supplement composition obtained at step (a) is provided in the form of a liquid preparation and comprises the extract isolated by using a mixture of water and ethanol, in which mixture amount of ethanol is about 0.5 vol-%.

3. The method of claim 1, wherein the feed supplement composition obtained at step (a) is provided in the form of a dry preparation provided as a powder or a particulate, said composition comprises the extract isolated by using a mixture of water and ethanol, in which mixture amount of ethanol is about 70 vol-%.

4. The method of claim 3, wherein the dry feed supplement composition obtained at step (a) is further admixed to a suitable liquid carrier prior to performing the step (b).

5. The method of claim 4, wherein the dosage regimen includes administering the feed supplement composition to poultry via drinking water in daily amount of 0.2-6 g per kg of live weight.

6. The method of claim 2, wherein the dosage regimen includes administering the feed supplement composition to poultry via drinking water in daily amount of 2.7-6 g per kg of live weight.

7. The method of claim 3, wherein the dosage regimen includes administering the feed supplement composition to poultry via the basal feed formula in daily amount of 0.3-60 mg per kg of live weight.

8. The method of claim 1, wherein poultry is selected from the group consisting of: broilers, breeders, commercial layers at different age stage, and replacement chickens of breeder- and commercial layer categories.

9. The method of claim 3, wherein the dosage regimen includes administering the feed supplement composition to broiler chickens continuously during individual rearing periods, in particular, from the first day of life until slaughtering via the basal feed formula in daily amount of 0.3-60 mg per kg of live weight, as a substitution for in-feed antibiotics and coccidiostats, such as to improve growth performance and to increase live-weight gain attainable by enhanced feed palatability and increased feed intake, to improve feed conversion efficiency attainable by increased digestibility rates of feed nutrients, to enhance eviscerated. carcass yield And to improve meat quality attainable by reducing stress impact.

10. The method of claim 4, wherein the dosage regimen includes administering the feed supplement composition to broiler chickens during the first 3-5 days. of life via drinking water in daily amount of 0.2-6 g per kg of live weight, such as to improve growth performance and to increase live weight gain attainable by increased digestibility rates of feed nutrients.

11. The method of claim 2, wherein the dosage regiment includes administering the feed supplement composition to replacement. chickens and commercial layers at different age stages continuously during individual rearing periods or in short-term periods of 10-20 days via the basal feed formula in daily amount of 9-360 mg per kg of live weight, such as to improve laying performance and to enhance feed conversion efficiency attainable by increased digestibility rates of feed nutrients.

12. The method of claim 2, wherein the dosage regimen includes administering the feed. supplement. composition to layer breeders and broiler breeders at different age stages and to the corresponding replacement chickens continuously during individual rearing periods or in short-term periods of 10-20 days via the basal feed formula in daily amount of 9-360 mg per kg of live weight, such as to improve settable egg quality, to enhance feed conversion efficiency and to prevent disease outbreaks, feather pecking and cannibalism in poultry in conditions of stress impact during catching, moving and vaccination, and to improve reproductive performance after feed withdrawal induced molting.
